SEQUENCE LISTING (5 sequences)

SEQ ID NO: 1 - RNA, 20 nt, synthetic construct
  source 1..20: mol_type = other RNA
atgatggagcgccgctgttt

SEQ ID NO: 2 - DNA, 8552 bp, synthetic construct
  source 1..8552: mol_type = other DNA
cccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcagacgagggcagaggaagtcttctaacatgcggtgacgtggaggagaatcccggccctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacaattggatatccccgggaaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatactagtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatgatggagcgccgctgtttgttttagagctagaaatagcaagttaaaataaggctagtccgttatcaacttgaaaaagtggcaccgagtcggtgctttttttaagcacgcgttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatc

SEQ ID NO: 3 - DNA, 7614 bp, synthetic construct
  source 1..7614: mol_type = other DNA
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcaggatgaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatc

SEQ ID NO: 4 - DNA, 8599 bp, synthetic construct
  source 1..8599: mol_type = other DNA
acggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattccaaagccactcactcgtctgcctgccgctgacccccccaggcctctccaaagttcagcaaccaaagagtcaggcctggtacagaagggttgtcaggctgaggcccattctgctggttggtgcctgggcctgagtcaccatctagaacattcccaagtttgaactcaggtacggtgctcagtctctccaggaatcagtttcaacaagataatcctttgctcacccttctcatggaattaaatgaggctgtctgcaaatgtcatttgaaaactgctaacctttttgggatcatttaaaaggcagaactgaaacatgcttcttcacccctctgaacccctggctctgcaagttcctctgcttccgcagctattccatccccagataaagacttgaggtgtggagaggatagagggcctgctaccttgagaacttcccactcagggcagcgacctctgtacacggtcatttcaagcacacctggtctcagtgttcattgtttatgttccccccgaccccaccctgggcacatacattttcctgctccatgagggatccttgtccctgccctccctgccctgtagagataggagttgctcttacttacttgatcaaatacacatcatacttcccagcagagcggccagatttcctttgcttaagcttccgtgtccagccttcaggcagggtggggtcatcatacatgggtccccggtcacggatgatggagcgccgctgtttgggggaggcagaagcttccggcacagccggggcggagcctgacccttctgatgtctctgctttgcctgcctctgcgggctcagcagagtggtgggctgatggctgcacgggctcatgcttgccctctttctcttctttcttatctttcttcacctttttaaacttgaggggtttgtccttgaggccctggaggtcctggtcttctgacttttcttccctgaagtgttaaacaagtatgtaagtatcacagagaacatgccagtctgcagaacaagtgagcagaggcaggcttggacccccaccacatgcaaggccccaggccatcatacactcagatcttgatgtccttccccaggacaacagagactcgggccaagctgggacccaaatatttctttaggaatttgggctgtgggtggtgctggccggtgtgcctgacctgcactgtggccacccacaagtctcctagcactgcaggagaatcagagccctttcctctgagcggaaacactgcggctcctgagcataccaccccagcatgagtcgcacttcaagggactctgggctcgatggctgacaagaggccgtcgactgctggaaagggagccaaatgggggaggggtctcctttgtgaaggccgggccttgggtgtgtttttggttgcggccgcaaaaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 1399 bp, Homo sapiens
  source 1..1399: mol_type = genomic DNA
aaccaaaaacacacccaaggcccggccttcacaaaggagacccctcccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctggggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaagcaaagaaggagtattatccgtgaccggggacccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcaccgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttg

(19) United States
(12) Patent Application Publication
(10) Pub. No.: US 2024/0424139 A1
(43) Pub. Date: Dec. 26, 2024

(54) Magnetic-Assisted Nanoparticle Delivery and Gene Editing Systems and Methods of Use

(71) Applicant: Rutgers, The State University of New Jersey, New Brunswick, NJ (US)

(72) Inventors: Ki-Bum Lee, Monmouth Junction, NJ (US); Hyeon-Yeol Cho, Piscataway, NJ (US); Myung-Sik Yoo, New Brunswick, NJ (US)

(73) Assignee: Rutgers, The State University of New Jersey, New Brunswick, NJ (US)

(21) Appl. No.: 18/751,997

(22) Filed: Jun. 24, 2024

Related U.S. Application Data
(60) Provisional application No. 63/510,217, filed on Jun. 26, 2023.

(51) Int. Cl. A61K 48/00; A61K 9/51; A61K 38/17; B82Y 5/00; C12N 9/22; C12N 15/11; C12N 15/90

(52) U.S. Cl. CPC A61K 48/005; A61K 9/5115; A61K 9/5146; A61K 38/1709; A61K 38/465; C12N 9/22; C12N 15/111; C12N 15/907; B82Y 5/00; C12N 2310/20

(57) ABSTRACT

This disclosure provides a novel magnetic nanoparticle-assisted genome editing (MAGE) platform, which significantly improves transfection efficiency and biocompatibility in stem cells as well as the efficiency of CRISPR-Cas systems. Due to the great potential of CRISPR-Cas systems, the disclosed MAGE platform can be used for a host of applications in stem cell therapy for genetic disorders.

### CROSS-REFERENCE TO RELATED APPLICATIONS

This application claims priority under 35 U.S.C. § 119(e) to U.S. Provisional Patent Application No. 63/510,217, filed Jun. 26, 2023. The foregoing application is incorporated by reference herein in its entirety.

### STATEMENT REGARDING FEDERALLY SPONSORED RESEARCH

This invention was made with government support under grant number 1R21NS085569-01 awarded by the National Institutes of Health. The government has certain rights in the invention.

### REFERENCE TO A SEQUENCE LISTING

This application incorporates by reference the Sequence Listing submitted in Computer Readable Form as a xml file named “Sequence listing 070439.01830.xml” created on May 28, 2024 and containing 31,576 bytes.

### FIELD OF THE INVENTION

This disclosure relates generally to magnetic-assisted nanoparticle delivery and gene editing systems and methods of use and methods of use thereof.

### BACKGROUND OF THE INVENTION

The discovery and use of clustered, regularly interspaced, short palindromic repeats (CRISPR)-associated endonuclease, e.g., Cas9, marks a significant breakthrough in the field of genetic engineering, with its ability to target and cut DNA at specific genomic loci in mammalian cells using a single guide RNA (gRNA). As a result, it has been successfully applied to various genome editing applications, including the genetic knock-out, knock-in, and correction of mutated genes, all of which can have significant therapeutic implications (Cong, L. et al. Science 339, 819-823 (2013)).

Although the essential components of the CRISPR/Cas9 system—Cas9, gRNA, and donor DNA—are usually delivered using viral methods such as adeno-associated viral (AAV) and lentiviral vectors, it is known that these viral vectors can have limited packing abilities and can provoke mutagenesis or carcinogenesis. As such, there has been increasing interest in the development of non-viral delivery methods as an alternative, including the use of nano-/microcarriers composed of lipid or cationic polymers. Compared with traditional viral methods, these non-viral methods have unique advantages, including the ability to deliver larger genetic payloads with lower immunogenicity as well as avoiding the issue of endogenous virus recombination, minimizing the chance of short-term and long-term adverse effects (Yin, H. et al. Nat Rev Genet 15, 541-555 (2014)). However, despite these advantages, non-viral delivery methods are plagued by low delivery efficiencies that must be overcome before they can be translated to the clinic.

Given the above challenges, there is a pressing need for more effective gene delivery and editing systems.

### SUMMARY OF THE INVENTION

This disclosure addresses the need mentioned above in a number of aspects. In one aspect, this disclosure provides a magnetic-assisted nanoparticle delivery and gene editing system. The system comprises: (a) a magnetic core-shell nanoparticle (MCNP) having a magnetic core and a negatively-charged shell (e.g., silica shell) coated on the magnetic core; (b) an inner cationic polymer layer disposed on the surface of the negatively-charged shell; (c) a multi-plasmid layer disposed on the inner cationic polymer layer, the multi-plasmid layer comprising one or more polynucleotides, wherein the one or more polynucleotides are associated through electrostatic interactions with the inner cationic polymer layer; and (d) an outer cationic polymer layer disposed on the multi-plasmid layer, wherein the outer cationic polymer layer is configured to encapsulate and protect the multi-plasmid layer and improve intracellular delivery/releasing efficiency of the one or more polynucleotides in a cell.

In some embodiments, the magnetic core comprises magnetic particles, e.g., ZnFe₂O₄. In some embodiments, the negatively-charged shell is a silica shell. In some embodiments, the inner cationic polymer layer or the outer cationic polymer layer comprises branched polyethyleneimine (PEI). In some embodiments, the PEI is a tetramethylrhodamine (TRITC)-labeled PEI. In some embodiments, the PEI has a molecular weight of about 10 kDa. In some embodiments, the inner cationic polymer layer and the outer cationic polymer layer may include the same or different cationic polymers.

In some embodiments, the system has a polynucleotide:MCNP mass ratio of about 1:10 to about 1:20. In some embodiments, the system has a polynucleotide:MCNP mass ratio of about 1:15.

In some embodiments, the magnetic core has a diameter of about 6 nm to about 10 nm. In some embodiments, the silica shell has a thickness of about 20 nm to about 30 nm. In some embodiments, the MCNP has an average diameter of about 80 nm to about 110 nm. In some embodiments, the MCNP has an average hydrodynamic size of about 95 nm to about 105 nm. In some embodiments, the MCNP has a zeta potential of about 18 mV to about 28 mV.

In some embodiments, the polynucleotides comprise a first polynucleotide and a second polynucleotide that is different from the first polynucleotide. In some embodiments, the first polynucleotide or the second polynucleotide comprises a plasmid. In some embodiments, the plasmid is linearized, e.g., by a restriction enzyme.

In some embodiments, the first polynucleotide comprises a CRISPR-associated (Cas) gene. In some embodiments, the first polynucleotide further comprises a gRNA (or guide RNA) sequence comprising a crRNA and optionally a trans-activating CRISPR RNA (tracrRNA). In some embodiments, the second polynucleotide comprises a donor DNA. In some embodiments, the polynucleotides comprise a third polynucleotide comprising a gRNA sequence comprising a crRNA and optionally a tracrRNA. In some embodiments, the Cas gene may include Cas9 (Csn1), Cas12a (Cpf1), Cas13a (C2c2), and Cas13b (C2c6).

In some embodiments, the cell comprises a eukaryotic cell, such as a plant, animal, or human cell. In some embodiments, the cell comprises a stem cell, e.g., an iPSC-derived neural progenitor cell (iPSC-NPC). In some embodiments, the donor DNA comprises a methyl CpG binding protein 2 (MECP2) gene.

Also provided in this disclosure are (i) a composition and (ii) a kit, comprising the system, as described above.

In another aspect, this disclosure also provides a method of modifying a target sequence of interest in a cell. The method comprises delivering the system or the composition, as described above, to a cell containing the target sequence.

In yet another aspect, the disclosure further provides a method of treating a disease of a subject caused by a genetic defect in a target sequence. The method comprises administering the system or the composition, as described above, to a cell containing the target sequence in a subject in need thereof.

In some embodiments, the target sequence is located at genomic loci of interest. In some embodiments, the target sequence comprises DNA.

In some embodiments, the target sequence is associated with a disease. In some embodiments, the disease is caused by a genetic defect in the target sequence. In some embodiments, the disease is cancer. In some embodiments, the disease is Rett syndrome (RTT).

In some embodiments, the cell comprises a eukaryotic cell. In some embodiments, the cell comprises a plant, animal, or human cell. In some embodiments, the cell comprises a stem cell, e.g., an iPSC-NPC.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1i, 1C, and 1D (collectively “FIG. 1”) are a set of diagrams showing a magnetic nanoparticle-assisted gene editing (MAGE) platform for repairing Rett syndrome from patient-derived stem cells with non-viral CRISPR-Cas9 delivery. FIG. 1 shows an example of MAGE that is composed of a magnetic nanoparticle, two plasmids of CRISPR-Cas9 and donor DNA for gene editing, and cationic polymers. FIG. 1B shows structural information of the MAGE platform before and after loading plasmids for intracellular delivery. FIG. 1C shows that MAGEs are delivered to Rett syndrome patient-derived induced-neuronal progenitor cells (iPS-NPCs) possessing the MeCP2 gene mutation via magnetofection. MAGE-transfected cells were sorted by magnetic-activated cell sorting (MACS) to collect the iPSC-NPCs that possess the plasmids. FIG. 1D shows that the intracellularly delivered MAGEs released two plasmids designed for different purposes; pCas9-gRNA-Puro for Cas9 protein with gRNA expression and pDonor-MeCP2-EGFP for editing of mutated MeCP2 via homologous repair. The MeCP2 repaired neuron shows morphological improvements as bigger soma size and more dendrites with functional MeCP2 proteins.

FIGS. 2A, 2B, 2C, 2D, and 2E (collectively “FIG. 2”) are a set of diagrams showing layer-by-layer conjugation of MAGE. FIG. 2A is a schematic diagram of the MAGE synthesis process. FIGS. 2B and 2C are TEM images of the magnetic core-mesoporous silica shell nanoparticle (FIG. 2B) before and (FIG. 2C) after the plasmid loading. FIG. 2D shows that size of the particle was increased through the synthesis process. FIG. 2E shows that each step of the synthesis process was confirmed by surface charge change with each layer's unique electrostatic information; silica shell surface (negative), PEI (positive), and plasmids (negative).

FIGS. 3A, 3B, 3C, 3D, and 3E (collectively “FIG. 3”) are a set of diagrams showing that MAGE delivered cell sorting and its gene expression. FIG. 3A shows the strategy for MAGE-mediated gene editing of RTT-NPCs. White dash-line circles indicate magnetically captured MAGE containing cells. Scale bar: 100 μm. FIG. 3B is a confocal microscopic image of RTT-NPC 24 hours after MAGE transfection. Rhodamine B labeled MAGE is highlighted. Scale bar: 20 μm. FIG. 3C shows quantification of nanoparticle delivered cells population (t-test, ***P<0.001, error bars: s.e.m.). FIG. 3D shows cytotoxicity analysis of MAGE transfected cells (t-test, **P<0.01, error bars: s.e.m.). FIG. 3E shows quantification of EGFP expressed cells population (DAPI staining) compared to control 24 hours after MAGE transfection (t-test, *P<0.05, error bars: s.e.m.). MF, magnetofection, MACS, magnetic-assisted cell sorting; MAGE, magnetic nanoparticle-assisted gene editing; RTT-NPCs, Rett syndrome patient-derived neuron progenitor cells; EGFP, enhanced green fluorescence protein.

FIGS. 4A, 4B, 4C, and 4D (collectively “FIG. 4”) are a set of diagrams showing correction of mutated MeCP2 gene by non-viral delivered CRISPR-Cas9. FIG. 4A is a graphical representation of the HDR results with various working conditions. Box of the unmodified sequence indicates the mutated sequence. FIG. 4B shows a GCD assay for 3 representative off-target candidate genes to confirm the off-target effect of the MeCP2-gRNA. FIG. 4C shows the results of the Western blot of MeCP2 protein expression two weeks after the transfection and quantification of MeCP2 levels in differentiated cells. FIG. 4D shows the results of immunostaining of differentiated neurons after MAGE-mediated MeCP2 editing. Edited RTT-neurons express the MeCP2 in the nuclei as same as WT-neurons, while MeCP2 is not shown in control RTT-neurons. The white arrow indicates MeCP2+ cells. Scale bar: 20 μm.

FIGS. 5A, 5B, 5C, 5D, 5E, 5F, 5G, and 5H (collectively “FIG. 5”) are a set of diagrams showing improved phenotypes and functional recovery of edited RTT-neuron. FIGS. 5A, 5B, and 5C show microscopic images of differentiated WT-neurons (FIG. 5A), control RTT-neurons (FIG. 5B), and edited RTT-neurons (FIG. 5C). Scale bar: 20 μm. FIGS. 5D, 5E, and 5F show phenotypic analysis of edited RTT-neurons in the length of neurites (FIG. 5D), soma size (FIG. 5E), and the number of neurites per cell (FIG. 5F) compared to WT-neurons and control RTT-neurons. FIGS. 5G and 5H show a calcium response of edited RTT-neurons monitored with time-lapse imaging (FIG. 5G) and fluorescence intensity recording (FIG. 5H) and comparison with control RTT-neurons. Edited RTT-neuron indicated in yellow arrow and control RTT-neuron indicated in white arrow. Scale bar: 10 μm.

### DETAILED DESCRIPTION OF THE INVENTION

The use of CRISPR-Cas systems to correct mutations holds great promise for the treatment of multiple diseases, including Rett syndrome. However, low delivery efficiencies hinder their translation. To address this problem, this disclosure provides a magnetic nanoparticle-assisted genome editing (MAGE) platform, which significantly improves transfection efficiency and biocompatibility in stem cells as well as the efficiency of CRISPR-Cas systems. This innovative platform offers a number of advantages, such as (i) faster and more efficient delivery of multiple genes due to magnetofection, (ii) magnetic-activated cell sorting (MACS), (iii) enhanced biocompatibility, and (iv) in-situ, real-time tracking of the delivery process, which all synergistically enhance the gene repairing efficiency of CRISPR-Cas systems. Owing to the great potential of CRISPR-Cas systems, the implications of the disclosed MAGE system go well beyond the treatment of Rett syndrome and can be used for a host of applications in stem cell therapy for genetic disorders.

### Magnetic Nanoparticle-Assisted Genome Editing (MAGE) Systems

In one aspect, this disclosure provides a magnetic-assisted nanoparticle delivery and gene editing system. The system comprises: (a) a magnetic core-shell nanoparticle (MCNP) having a magnetic core and a negatively-charged shell (e.g., silica shell (e.g., tetraethyl orthosilicate (TEOS)), poly(acrylic acid) (PAA), carboxylic acid modified poly(vinyl alcohol) (PVA), polyvinylpyrrolidone (PVP)) coated on the magnetic core; (b) an inner cationic polymer layer disposed on the surface of the negatively-charged shell; (c) a multi-plasmid layer disposed on the inner cationic polymer layer, the multi-plasmid layer comprising one or more polynucleotides, wherein the one or more polynucleotides are associated through electrostatic interactions with the inner cationic polymer layer; and (d) an outer cationic polymer layer disposed on the multi-plasmid layer, wherein the outer cationic polymer layer is configured to encapsulate and protect the multi-plasmid layer and improve intracellular delivery/releasing efficiency of the one or more polynucleotides in a cell.

### Magnetic Core Shell Nanoparticle (MCNP)

The term “magnetic particles” refers to magnetically responsive particles that contain one or more metals or oxides or hydroxides thereof. Such particles typically react to a magnetic force resulting from a magnetic field. The field can attract or repel the particle towards or away from the source of the magnetic field, respectively, optionally causing acceleration or movement in a desired direction in space. A magnetically detectable nanoparticle is a magnetic particle that can be detected within a living cell as a consequence of its magnetic properties. Magnetic particles may comprise one or more ferrimagnetic, ferromagnetic, paramagnetic, and/or superparamagnetic materials. Useful particles may be made entirely or in part of one or more materials selected from the group consisting of: iron, cobalt, nickel, niobium, magnetic iron oxides, hydroxides such as ZnFe₂O₄, maghemite (γ-Fe₂O₃), magnetite (Fe₃O₄), feroxyhyte (FeO(OH)), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the aforementioned oxides or hydroxides, and mixtures of any of the foregoing. Additional materials that may be used in magnetic particles include yttrium, europium, and vanadium.

A magnetic nanoparticle may contain a magnetic material and one or more non-magnetic materials, which may be a metal or a nonmetal. In some embodiments, the nanoparticle is a composite nanoparticle comprising an inner core or layer containing a first material and an outer layer or shell containing a second material, wherein at least one of the materials is magnetic. In some embodiments, the nanoparticle is an iron oxide nanoparticle, e.g., the nanoparticle has a core of iron oxide. Optionally the iron oxide is monocrystalline. In some embodiment, the nanoparticle is a superparamagnetic iron oxide nanoparticle, e.g., the nanoparticle has a core of superparamagnetic iron oxide. In some embodiments, the nanoparticle has a core of ZnO₄.

The magnetic core may have various sizes. For example, the magnetic core may have a diameter of about 6 nm to about 10 nm.

In some embodiments, the nanoparticle comprises silica (SiO₂). For example, the nanoparticle may consist at least in part of silica, e.g., it may consist essentially of silica or may have an optional coating layer composed of a different material. In some embodiments, the MCNP has a magnetic nanoparticle core and an outside silica coating layer (e.g., inert mesoporous silica shell). The amount of silica in the nanoparticle, e.g., in a coating layer comprising silica, can range from approximately 5% to 100% by mass, volume, or number of atoms, or can assume any value or range between 5% and 100%. Silica-containing nanoparticles may be made by a variety of methods. Certain of these methods utilize the Stδber synthesis, which involves hydrolysis of tetraethoxyorthosilicate (TEOS) catalyzed by ammonia in water/ethanol mixtures, or variations thereof. Microemulsion procedures can be used. For example, a water-in-oil emulsion in which water droplets are dispersed as nanosized liquid entities in a continuous domain of oil and surfactants and serve as nanoreactors for nanoparticle synthesis offer a convenient approach. Silica nanoparticles can be functionalized with biomolecules such as polypeptides and/or “doped” or “loaded” with certain inorganic or organic fluorescent dyes (see, e.g., U.S. Patent Publication 2004/0067503; Bagwe et al., 2004, Langmuir, 20:8336; Van Blaaderen and Vrij, 1992, Langmuir, 8:2921; Lin et al., 2005, J. Am. Chem. Soc, 17:4570; Zhao et al., 2004, Adv. Mat, 16: 173; and Wang et al., 2005, Nano Letters, 5:37; all of which are incorporated herein by reference).

The silica shell can be a mesoporous silica shell. The fact that the shell is referred to as a silica shell does not preclude materials other than silica from also being incorporated within the silica shell. In some embodiments, the silica shell may be substantially spherical with a plurality of pore openings through the surface, providing access to the pores. However, the silica shell can have shapes other than substantially spherical shapes in other embodiments of the present invention. Generally, the silica body defines an outer surface between the pore openings, as well as sidewalls within the pores. The pores can extend through the silica shell to another pore opening or can extend only partially through the silica shell such that it has a bottom surface of the pore defined by the silica shell.

In some embodiments, the silica shell is mesoporous. In other embodiments, the silica shell is microporous. As used herein, “mesoporous” means having pores with a diameter between 2 nm and 50 nm, while “microporous” means having pores with a diameter smaller than 2 nm. In general, the pores may be of any size. In some embodiments, the pores are substantially cylindrical.

The silica shell may have varying thickness, such as between about 20 nm and about 30 nm.

### Size

MCNPs can have a variety of different shapes including spheres, oblate spheroids, cylinders, ovals, ellipses, shells, cubes, cuboids, cones, pyramids, rods (e.g., cylinders or elongated structures having a square or rectangular cross-section), tetrapods (particles having four leg-like appendages), triangles, prisms, etc. Also disclosed herein are populations of MCNPs, in which the nanoparticles of at least about 10%, 20%, 30%, 40%, 50% 60%, 70%, 80% or 90% of the MCNPs can be about 50 nm to about 130 nm (e.g., about 60 nm to about 120 nm, about 70 nm to about 110 nm, about 80 nm to about 110 nm) in their largest dimension.

In some embodiments, MCNPs have an average diameter of about 50 nm to about 120 nm (e.g., about 60 nm to about 120 nm, about 70 nm to about 110 nm, about 80 nm to about 110 nm).

In some embodiments, MCNPs have an average hydrodynamic size of about 90 nm to about 110 nm (e.g., about 95 nm to about 115 nm, about 95 nm to about 110 nm, about 95 nm to about 105 nm).

The MCNPs according to some embodiments of the current invention are referred to as nanoparticles. The term “nanoparticles,” as used herein, is intended to include particles as large as 1000 nm. In general, particles larger than 300 nm become ineffective in entering living cells. In that case, larger particles can tend to settle rather than remaining suspended in Brownian motion. As used herein, size of the MCNPs refers to the size of the primary particles, as measured by transmission electron microscopy (TEM) or a similar visualization technique.

It may be desirable to use a population of MCNPs that is relatively uniform in terms of size, shape, and/or composition so that each particle has similar properties. For example, at least 80%, at least 90%, or at least 95% of the particles may have a diameter or largest dimension that falls within 5%, 10%, or 20% of the average diameter or largest dimension. In some embodiments, a population of particles may be heterogeneous with respect to size, shape, and/or composition.

In some embodiments, one or more substantially uniform populations of MCNPs are used, e.g., 2, 3, 4, 5, or more substantially uniform populations having distinguishable properties (e.g., optical and/or magnetic properties). Each population of particles is associated with an agent. Use of multiple distinguishable particle populations allows tracking of multiple different agents concurrently. It will be appreciated that a combination of two or more populations having distinguishable properties can be considered to be a single population.

### Charges

Zeta potential is a measurement of surface potential of a particle. In some embodiments, the MCNP has a zeta potential of about 18 mV to about 28 mV (e.g., about 20 mV to about 26 mV, about 22 mV to about 24 mV). Zeta potential in solution can be measured, for example, by ZetaSizer Nano (Malvern Instruments Ltd., Worcestershire, UK). Electrophoretic mobility is used as an approximation of particle surface charge and can be used to calculate zeta potential. The Helmholtz-Smoluchowski equation was used to recalculate electrophoretic mobility into zeta potential.

### Cationic Polymer Layers

As used herein, the term “polymer” is a macromolecule composed of repeating structural units, usually in a linear or branched sequence.

The cationic polymer may be any polymer bearing an overall positive charge. Examples of cationic polymers may include, without limitation, polyethyleneimine (PEI), polyamidoamine, polylysine, poly(allylamine) or poly(diallyldimethylammonium chloride). Other cationic polymers will be apparent to those of skill in the art, and may be found, for example, in “Polymer Handbook, 4th Edition, Edited by: Brandrup et al.; John Wiley & Sons, 1999; and De Smedt et al., Pharmaceutical Research, vol. 17, no. 2, pp. 113-126). Other examples include, chitosan, poly(N-isopropyl acrylamide-co-Acrylamide), Poly(N-isopropyl acrylamide-co-acrylic acid), poly(L-lysine), diethylaminoethyl-dextran, poly-(N-ethyl-vinylpyridinium bromide), poly(2-di-methylamino)ethyl methacrylate), poly(ethylene glycol)-co-poly(trimethylaminoethylmethacryl-ate chloride). Cationic polymer modified nanoparticles have a positive charge. In some embodiments, the cationic polymer is PEI.

In some embodiments, the cationic polymer has a weight average molecular weight less than about 30 kDa, less than about 25 kDa, less than about 20 kDa, less than about 15 kDa, or less than about 10 kDa. In some embodiments, the cationic polymer has a weight average molecular weight greater than about 600 Da, greater than about 1 kDa, greater than about 1.5 kDa, or greater than about 1.8 kDa, greater than about 2 kDa, greater than about 3 kDa, or greater than about 4 kDa, or greater than about 5 kDa. The range of molecular weight may be between any recited endpoints.

In some embodiments, the inner cationic polymer layer or the outer cationic polymer layer comprises branched PEI. In some embodiments, the PEI is a tetramethylrhodamine (TRITC)-labeled PEI. In some embodiments, the PEI has a molecular weight of about 10 kDa or about 25 kDa. In some embodiments, the inner cationic polymer layer and the outer cationic polymer layer may include the same or different cationic polymers.

The cationic polymer may be bound covalently or electrostatically to the surface of the silica shell. In some embodiments, the cationic polymer is electrostatically bound to the surface of the silica shell. For example, the cationic polymer may bind electrostatically to the silica shell, which has an overall negative charge.

As used herein, “electrostatically bonded” means bonded based on the attraction of opposite charges. An unmodified nanoparticle has a negative charge, due to the presence of free silyl hydroxide residues on the surface of the nanoparticle. The particle may also bear a surface modification having a negative charge (such as a phosphonate modification), such that the overall charge of the surface is negative. The surface may be modified with material bearing a positive charge, which will bind to the surface electrostatically.

### Multi-Plasmid Layer Containing Polynucleotide(s)

A “polynucleotide” or “nucleic acid” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

In some embodiments, the disclosed polypeptide can be encoded by a codon-optimized sequence. For example, the nucleotide sequence encoding the polypeptide may be codon-optimized for expression in a eukaryote or eukaryotic cell. In some embodiments, the codon-optimized polypeptide is codon-optimized for operability in a eukaryotic cell or organism, e.g., a yeast cell, or a mammalian cell or organism, including a mouse cell, a rat cell, and a human cell or non-human eukaryote organism.

Generally, codon optimization refers to a process of modifying a nucleic acid sequence to enhance expression in the host cells by substituting at least one codon of the native sequence with codons that are more frequently or most frequently used in the genes of that host cell while maintaining the native amino acid sequence. Various species exhibit a particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.). In some embodiments, one or more codons (e.g., 1, 2, 3, 4, 5, 10, 15, 20, 25, 50, or more, or all codons) in a sequence encoding the polypeptide corresponds to the most frequently used codon for a particular amino acid. As to codon usage in yeast, reference is made to the online Yeast Genome database available at http://www.yeastgenome.org/community/codonusage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11; as well as Codon usage in plant genes, Murray et al., Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

In some embodiments, the polynucleotides can be plasmids. In some embodiments, the polynucleotides may include a first polynucleotide and a second polynucleotide that is different from the first polynucleotide. In some embodiments, the first polynucleotide or the second polynucleotide comprises a plasmid. In some embodiments, the polynucleotides can be relaxed or coiled (e.g., supercoiled). In some embodiments, the plasmid is linearized, e.g., by a restriction enzyme.

In some embodiments, the polynucleotides may include polynucleotides encoding one or more components of a CRISPR-Cas system, e.g., Cas protein, guide RNA, donor DNA. For example, the first polynucleotide comprises a Cas gene or a variant thereof. In some embodiments, the first polynucleotide further comprises a gRNA sequence comprising a crRNA and optionally a tracrRNA. In some embodiments, the second polynucleotide comprises a donor DNA. In some embodiments, the Cas gene and the guide RNA may be carried on separate polynucleotides. Accordingly, the polynucleotides comprise a third polynucleotide comprising a gRNA sequence comprising a crRNA and optionally a tracrRNA. In some embodiments, the Cas gene may include Cas9 (Csn1), Cas12a (Cpf1), Cas13a (C2c2), and Cas13b (C2c6). In some embodiments, the Cas gene can be one of SpCas9, dCas9, Cas nickase, Cas9 nickase, FnCas9, StCas9, SaCas9, LpCas9, FnCas12, Cas12 nickase, AsCas12, LbCas12, Cas12a, Cas12b, Cas12c, Cas13, and Cas 13d.

In some embodiments, the system has a polynucleotide:MCNP mass ratio of about 1:10 to about 1:20 (e.g., about 1:10, about 1:11, about 1:12, about 1:13, about 1:14, about 1:15, about 1:16, about 1:17, about 1:18, about 1:19, about 1:20). In some embodiments, the system has a polynucleotide:MCNP mass ratio of about 1:15.

In some embodiments, the cell comprises a eukaryotic cell, such as a plant, animal, or human cell. In some embodiments, the cell comprises a stem cell, e.g., an iPSC-derived neural progenitor cell (iPSC-NPC). In some embodiments, the donor DNA comprises a methyl CpG binding protein 2 (MECP2) gene.

In some embodiments, the polynucleotides bind electrostatically to the inner and outer cationic polymer layers and, in some circumstances, the outside surface of the silica shell. The polynucleotides are encapsulated and protected by the outer cationic polymer layer, thus improving intracellular delivery/releasing efficiency of the one or more polynucleotides in a cell. Without wishing to be bound by theory, the cationic polymer envelopes and protects the polynucleotides from degradation. Polynucleotide-containing structures according to the invention may, for example, deliver polynucleotides to the interior of a cell when the MAGE structure enters the cell. Genes, e.g., components of CRISPR-Cas systems, may thus be successfully delivered into a cell.

### Compositions and Kits

### Pharmaceutical Compositions

The disclosed MAGE system can be incorporated into pharmaceutical compositions suitable for administration. The pharmaceutical compositions generally comprise the system and a pharmaceutically acceptable carrier in a form suitable for administration to a subject. Pharmaceutically-acceptable carriers are determined in part by the particular composition being administered, as well as by the particular method used to administer the composition. The pharmaceutical compositions are generally formulated as sterile, substantially isotonic and in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the nanoparticle construct, use thereof in the compositions is contemplated. Supplementary active compounds can also be incorporated into the compositions.

A pharmaceutical composition is formulated to be compatible with its intended route of administration. Solutions or suspensions used for intradermal or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as mannitol, sorbitol, sodium chloride in the composition. Prolonged absorption of the injectable compositions can be brought about by including in the composition a compound which delays absorption, e.g., aluminum monostearate and gelatin.

### Kits

This disclosure further provides kits containing reagents for performing the above-described methods, including, e.g., MCNPs, cationic polymers, polynucleotides encoding CRISPR:Cas components (e.g., Cas proteins, guide RNAs), and donor DNAs. In one embodiment, the kit may also include reagents for preparing MCNPs, such as ZnFe₂O₄, tetraethyl orthosilicate. In some embodiments, the kit may include polynucleotides (e.g., plasmids) encoding CRISPR proteins, such as Cas proteins, or one or more RNAs (e.g., guide RNA). In some embodiments, the kit can include one or more other reaction components. In such a kit, an appropriate amount of one or more reaction components is provided in one or more containers or held on a substrate.

Examples of additional components of the kits include, but are not limited to, one or more host cells, one or more reagents for introducing foreign nucleotide sequences into host cells, one or more reagents (e.g., probes or PCR primers) for detecting expression of the RNA or protein or verifying the target nucleic acid's status, and buffers or culture media for the reactions (in 1× or concentrated forms). The kit may also include one or more of the following components: supports, terminating, modifying or digestion reagents, osmolytes, and an apparatus for detection.

The reaction components used can be provided in a variety of forms. For example, the components (e.g., enzymes, RNAs, probes, and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits, it is preferred that they are provided and maintained below 0° C., preferably at or below −20° C., or otherwise in a frozen state.

A kit or system may contain, in an amount sufficient for at least one assay, any combination of the components described herein. In some applications, one or more reaction components may be provided in pre-measured single-use amounts in individual, typically disposable, tubes or equivalent containers. The amount of a component supplied in the kit can be any appropriate amount and may depend on the target market to which the product is directed. The container(s) in which the components are supplied can be any conventional container that is capable of holding the supplied form, for instance, microfuge tubes, microtiter plates, ampoules, bottles, or integral testing devices, such as fluidic devices, cartridges, lateral flow, or other similar devices.

The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for the use of the components.

### Methods and Uses

This disclosure also encompasses methods and uses of the MAGE systems described herein for modifying a target DNA sequence (e.g., a chromosomal sequence) or target RNA sequence, e.g., for altering or manipulating the expression of one or more genes or the one or more gene products, in prokaryotic or eukaryotic cells, in vitro, in vivo, or ex vivo. The disclosed MAGE systems (e.g., used to deliver CRISPR-Cas9 components to cells) provide an effective means for modifying (e.g., deleting, inserting, translocating, inactivating, activating) a target DNA (double-stranded, linear or supercoiled) in a multiplicity of cell types. Thus, the disclosed MAGE systems have a broad spectrum of applications, e.g., gene therapy, drug screening, disease diagnosis, and prognosis.

### Methods of Modifying Expression of a Target Polynucleotide

In one aspect, the disclosure provides a method of modifying expression of a target polynucleotide (e.g., target sequence of interest) in a eukaryotic cell. In some embodiments, the method allows a CRISPR-Cas complex (e.g., Cas protein/gRNA complex) to bind to the target polynucleotide, resulting in increased or decreased expression of the target polynucleotide or a gene comprising the target polynucleotide. In some embodiments, the CRISPR-Cas complex comprises Cas9 complexed with a gRNA sequence hybridized to a target sequence within the polynucleotide.

In some embodiments, the modification comprises cleaving one or two strands at the location of the target sequence by a Cas protein. In some embodiments, the modification results in decreased or increased transcription of a target gene. In some embodiments, the method further comprises repairing the cleaved target polynucleotide by homologous recombination with an exogenous template polynucleotide, wherein the repair results in a mutation comprising an insertion, deletion, or substitution of one or more nucleotides of the target polynucleotide. In some embodiments, the mutation results in one or more amino acid changes in a protein expressed from a gene comprising the target sequence. In some embodiments, the modification takes place in the eukaryotic cell in cell culture. In some embodiments, the method further comprises isolating the eukaryotic cell from a subject prior to the modification. In some embodiments, the method further comprises returning the eukaryotic cell and/or cells derived therefrom to the subject.

In some embodiments, the method of modifying a target polynucleotide comprises delivering the system or the composition, as described above, to a target sequence or a cell containing the target sequence. In some embodiments, following formation of a complex between the gRNA and the CRISPR-Cas protein and hybridization of the crRNA to one or more nucleic acids of the target sequence, the CRISPR-Cas protein induces a modification (e.g., cleavage) of the target sequence.

The target polynucleotide has no sequence limitation except that the sequence is followed by or preceds a PAM sequence. Other examples of PAM sequences are given above, and the skilled person will be able to identify further PAM sequences for use with a given CRISPR protein. The target polynucleotide can be in the coding region of a gene, in an intron of a gene, in a control region between genes, etc. The gene can be coding or non-coding.

The target polynucleotide can be any polynucleotide endogenous or exogenous to the cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide).

The method further comprises maintaining the cell or embryo under appropriate conditions such that the gRNA guides the Cas protein to the targeted site in the target sequence to modify the target sequence. In general, the cell can be maintained under conditions appropriate for cell growth and/or maintenance. Suitable cell culture conditions are well known in the art and are described, for example, in Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001), Santiago et al. (2008) PNAS 105:5809-5814; Moehle et al. (2007) PNAS 104:3055-3060; Urnov et al. (2005) Nature 435:646-651; and Lombardo et al. (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

An embryo can be cultured in vitro (e.g., in cell culture). Typically, the embryo is cultured at an appropriate temperature and in appropriate media with the necessary O₂/CO₂ ratio to allow the expression of the proteins and RNA scaffold, if necessary. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into a uterus of a female host. Generally speaking, the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and can result in a live birth of an animal-derived from the embryo. Such an animal would comprise the modified chromosomal sequence in every cell of the body.

### Methods of Generating a Model Eukaryotic Cell

In one aspect, this disclosure provides a method of generating a model eukaryotic cell comprising a mutated disease gene, which can be any gene associated with an increase in the risk of having or developing a disease. In some embodiments, the method comprises (a) introducing a system or a composition of the present invention into a eukaryotic cell; and (b) allowing a CRISPR-Cas/guide RNA complex (e.g., Cas9/gRNA complex, Cas12a/crRNA complex) to bind to a target polynucleotide to effect cleavage of the target polynucleotide within the disease gene, wherein the gRNA comprising the sequence that is hybridized to the target sequence within the target polynucleotide, thereby generating a model eukaryotic cell comprising a mutated disease gene.

In some embodiments, the cleavage comprises cleaving one or two strands at the location of the target sequence by a Cas protein. In some embodiments, the cleavage results in decreased or increased transcription of a target gene. In some embodiments, the method further comprises repairing the cleaved target polynucleotide by non-homologous end joining (NHEJ)-based gene insertion mechanisms with an exogenous template polynucleotide, wherein the repair results in a mutation comprising an insertion, deletion, or substitution of one or more nucleotides of the target polynucleotide. In some embodiments, the mutation results in one or more amino acid changes in protein expression from a gene comprising the target sequence.

A variety of eukaryotic cells are suitable for use in the method. For example, the cell can be a human cell, a non-human mammalian cell, a non-mammalian vertebrate cell, an invertebrate cell, an insect cell, a plant cell, a yeast cell, or a single-cell eukaryotic organism. A variety of embryos are suitable for use in the method. For example, the embryo can be a 1-cell, 2-cell, or 4-cell human or non-human mammalian embryo. Exemplary mammalian embryos, including one-cell embryos, such as mouse, rat, hamster, rodent, rabbit, feline, canine, ovine, porcine, bovine, equine, and primate embryos. In still other embodiments, the cell can be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells, and others. In exemplary embodiments, the cell is a mammalian cell or the embryo is a mammalian embryo. In some embodiments, the non-human mammal cell may include, but not limited to, primate bovine, ovine, porcine, canine, rodent, Leporidae such as monkey, cow, sheep, pig, dog, rabbit, rat or mouse cell. In some embodiments, the cell may be a non-mammalian eukaryotic cell, such as poultry bird (e.g., chicken), vertebrate fish (e.g., salmon) or shellfish (e.g., oyster, clam, lobster, shrimp) cell. In some embodiments, the non-human eukaryote cell is a plant cell. The plant cell may be of a monocot or dicot or of a crop or grain plant such as cassava, corn, sorghum, soybean, wheat, oat or rice. The plant cell may also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.).

### Methods of Developing a Biologically Active Agent

In another aspect, this disclosure provides a method for developing a biologically active agent that modulates a cell signaling event associated with a disease gene, which can be any gene associated with an increase in the risk of having or developing a disease. In some embodiments, the method comprises (a) contacting a test agent with a model cell, as described above; and (b) detecting a change in a readout that is indicative of a reduction or an augmentation of a cell signaling event associated with the mutation in the disease gene, thereby developing the biologically active agent that modulates the cell signaling event associated with the disease gene.

### Methods of Treatment

The above-described systems or compositions can be used in a therapeutic method of treatment. The therapeutic method of treatment may comprise gene or genome editing, or gene therapy. In one aspect, this disclosure provides a method of treating a subject in need thereof, comprising inducing gene editing by delivering to a cell in the subject a system or a composition of the present invention. In some embodiments, the method comprises inducing transcriptional activation or repression by delivering to a cell in the subject a system or a composition of the present invention.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

Many devastating human diseases have one common cause: genetic alteration or mutation. The disease-causing mutations in patients are either acquired through inheritance from their parents or are caused by environmental factors. These diseases include, but are not limited to, the following categories. First, some genetic disorders are caused by germline mutations. One example is cystic fibrosis, which is caused by mutations at the CFTR gene inherited from parents. A second suppressor mutation in the mutant CFTR can partially restore the function of CFTR protein in somatic tissues. Other example genetic diseases caused by a point genetic mutation that can be corrected by the disclosed technology include Gaucher's disease, alpha trypsin deficiency disease, sickle cell anemia, to name a few. Second, some diseases, such as chronic viral infectious diseases, are caused by exogenous environmental factors and resulting in genetic alterations. One example is AIDS, which is caused by insertion of the human HIV viral genome into the genome of infected T-cells. Third, some neurodegenerative diseases involve genetic alterations. One example is Huntington's disease, which is caused by expansion of CAG tri-nucleotide in the huntingtin gene of affected patients. Finally, cancers are caused by various somatic mutations accumulated in cancer cells. Therefore, correcting the disease-causing genetic mutations, or functionally correcting the sequence, provides an appealing therapeutic opportunity to treat these diseases.

Somatic genetic editing is an appealing therapeutic strategy for many human diseases. Through precise editing of the target DNA or RNA sequence, the CRISPR-Cas system can correct the mutated genes in genetic disorders, inactivate the viral genome in the infected cells, eliminate the expression of the disease-causing protein in neurodegenerative diseases, or silence the oncogenic protein in cancers. Accordingly, the system and method disclosed in this disclosure can be used in correcting underlying genetic alterations in diseases, including the above mentioned genetic disorders, chronic infectious diseases, neurodegenerative diseases, and cancer.

### Genetic Diseases

It is estimated that over six thousand genetic diseases are caused by known genetic mutations. Correcting the underlying disease-causing mutations in the pathological tissues/organs can provide alleviation or cure to the diseases. For example, cystic fibrosis affects 1 out of every 3,000 people in the US. It is caused by inheritance of a mutated CFTR gene, and 70% of the patients have the same mutation, deletion of a tri-nucleotide leading to a deletion of phenylalanine at position 508 (called Δ Phe 508). Δ Phe 508 leads to the mislocation and degradation of CFTR. The system and method disclosed in this invention can be used to convert a Val 509 residue (GTT) to Phe 509 (TTT) in affected tissues (lung), thereby functionally correcting the Δ Phe 508 mutation. In addition, a second suppressor mutation (such as R553Q or R553M or V510D) in the mutant Δ Phe 508 CFTR can partially restore the function of CFTR protein in somatic tissues.

Rett syndrome (RTT) is a genetic brain disorder that typically becomes apparent after 6 to 18 months of age in females. Symptoms include problems with language, coordination, and repetitive movements. Often there is slower growth, problems walking, and a smaller head size. Complications can include seizures, scoliosis, and sleeping problems. Those affected, however, may be affected to different degrees.

Rett syndrome is due to a genetic mutation of the MECP2 gene. This gene occurs on the X chromosome. Typically it develops as a new mutation, with less than one percent of cases being inherited from a person's parents. It occurs almost exclusively in girls. Boys who have a similar mutation typically die shortly after birth. Diagnosis is based on symptoms and can be confirmed with genetic testing.

There is no known cure for Rett syndrome. Treatment is directed at improving symptoms. Anticonvulsants may be used to help with seizures. Special education, physiotherapy, and braces may also be useful. Many people with the condition live into middle age.

### Chronic Infectious Diseases

The system and method, as disclosed, can also be used to specifically inactivate any gene in a viral genome that is incorporated into human cells/tissues. For example, the system and method disclosed in this invention allow one to create a stop codon for early termination of translation of the essential viral genes, and thereby remediate or cure the chronic debilitating infectious diseases. For example, current AIDS therapies can reduce viral load, but cannot totally eliminate dormant HIV from positive T cells. The system and method disclosed herein can be used to permanently inactivate one or two essential HIV gene expression in the integrated HIV genome in human T-cells by introducing one or two stop codons. Another example is the hepatitis B virus (HBV). The system and method disclosed here can be used to specifically inactivate one or two essential HBV genes, which are incorporated into the human genome, and silence HBV life-cycle.

### Neurodegenerative Diseases

Some neurodegenerative diseases are caused by gain-of-function mutations. For example, SOD1G93A leads to development of amyotrophic lateral sclerosis (ALS). The system and method disclosed in this invention can be used to either correct the mutation or eliminate the mutant protein expression by introducing a stop codon or by changing a splicing site.

### Diseases of the Muscular System

The present invention also contemplates delivering the CRISPR-Cas system described herein to muscle(s). Dystrophin is a cytoplasmic protein that provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein, which is over 3500 amino acids. Exon 51 is frequently adjacent to frame-disrupting deletions in DMD patients and has been targeted in clinical trials for oligonucleotide-based exon skipping. A clinical trial for the exon 51 skipping compound eteplirsen recently reported a significant functional benefit across 48 weeks, with an average of 47% dystrophin positive fibers compared to baseline. Mutations in exon 51 are ideally suited for permanent correction by NHEJ-based genome editing. The methods of US Patent Publication No. 20130145487, which relates to meganuclease variants to cleave a target sequence from the human dystrophin gene (DMD), may also be modified for the nucleic acid-targeting system of the present invention.

### Cancers

Many genes (including tumor suppressor genes, oncogenes, and DNA repair genes) contribute to the development of cancer. Mutations in these genes often lead to various cancers. Using the system and method disclosed herein, one can specifically target and correct these mutations. As a result, causative oncogenic proteins can be functionally annulled or their expression can be eliminated by introducing a point mutation at either the catalytic sites or splicing sites. In some embodiments, the treatment, prophylaxis or diagnosis of cancer is provided. The target is preferably one or more of the FAS, BID, CTLA4, PDCD1, CBLB, PTPN6, TRAC, or TRBC genes. Cancer may be one or more of lymphoma, chronic lymphocytic leukemia (CLL), B cell acute lymphocytic leukemia (B-ALL), acute lymphoblastic leukemia, acute myeloid leukemia, non-Hodgkin's lymphoma (NHL), diffuse large cell lymphoma (DLCL), multiple myeloma, renal cell carcinoma (RCC), neuroblastoma, colorectal cancer, breast cancer, ovarian cancer, melanoma, sarcoma, prostate cancer, lung cancer, esophageal cancer, hepatocellular carcinoma, pancreatic cancer, astrocytoma, mesothelioma, head and neck cancer, and medulloblastoma. This may be implemented with engineered chimeric antigen receptor (CAR) T cell. This is described in WO2015161276, the disclosure of which is hereby incorporated by reference and described herein below. Target genes suitable for the treatment or prophylaxis of cancer may include those described in WO2015048577, the disclosure of which is hereby incorporated by reference.

### Stem Cell Genetic Modification

In some embodiments, stem cell or progenitor cell can be genetically modified using the system and method disclosed in this invention. Suitable cells include, e.g., stem cells (adult stem cells, embryonic stem cells, iPS cells, etc.) and progenitor cells (e.g., cardiac progenitor cells, neural progenitor cells, etc.). Suitable cells include mammalian stem cells and progenitor cells, including, e.g., rodent stem cells, rodent progenitor cells, human stem cells, human progenitor cells, etc. Suitable host cells include in vitro host cells, e.g., isolated host cells.

In some embodiments, the present invention can be used for targeted and precise genetic modification of tissue ex vivo, correcting the underlying genetic defects. After the ex vivo correction, the tissues may be returned to the patients. Moreover, the technology can be broadly used in cell-based therapies for correcting genetic diseases.

### Genetic Editing in Animals and Plants

The system and method described above can be used to generate a transgenic non-human animal or plant having one or more genetic modification of interest. In some embodiments, the transgenic non-human animal is homozygous for the genetic modification. In some embodiments, the transgenic non-human animal is heterozygous for the genetic modification. In some embodiments, the transgenic non-human animal is a vertebrate, for example, a fish (e.g., zebrafish, goldfish, pufferfish, cavefish, etc.), an amphibian (frog, salamander, etc.), a bird (e.g., chicken, turkey, etc.), a reptile (e.g., snake, lizard, etc.), a mammal (e.g., an ungulate, e.g., a pig, a cow, a goat, a sheep, etc.; a lagomorph (e.g., a rabbit); a rodent (e.g., a rat, a mouse); or a non-human primate.

The invention can be used for treating diseases in animals in a way similar to those for treating diseases in humans, as described above. Alternatively, it can be used to generate knock-in animal disease models bearing specific genetic mutation(s) for purposes of research, drug discovery, and target validation. The system and method described above can also be used for introduction of point mutations to ES cells or embryos of various organisms, for the purpose of breeding and improving animal stocks and crop quality.

Methods of introducing exogenous nucleic acids into plant cells are well known in the art. Suitable methods include viral infection (such as double-stranded DNA viruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e., in vitro, ex vivo, or in vivo).

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product(s).” If the polynucleotide is derived from genomic DNA, expression may include splicing of the mRNA in a eukaryotic cell.

The terms “polypeptide,” “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open reading frame. In other words, a “fusion polypeptide” or “fusion protein” is a recombinant protein of two or more proteins which are joined by a peptide bond or via several peptides. The fusion protein may also comprise a peptide linker between the two domains.

The term “linker” refers to any means, entity or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, and reduction, etc., to provide a site for coupling. Methods for conjugation are well known by persons skilled in the art and are encompassed for use in the present invention. Linker moieties include, but are not limited to, chemical linker moieties, or for example, a peptide linker moiety (a linker sequence). It will be appreciated that modifications that do not significantly decrease the function of the RNA-binding domain and effector domain are preferred.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

As used herein, the term “derived from” refers to a process whereby a first component (e.g., a first molecule), or information from that first component, is used to isolate, derive or make a different second component (e.g., a second molecule that is different from the first). For example, the mammalian codon-optimized Cas polynucleotides are derived from the wild type Cas protein amino acid sequence. As used herein the term “wild type” is a term of the art understood by skilled persons and means the typical form of an organism, strain, gene, or characteristic as it occurs in nature as distinguished from mutant or variant forms.

As used herein, the term “variant” refers to a first composition (e.g., a first molecule) that is related to a second composition (e.g., a second molecule, also termed a “parent” molecule). The variant molecule can be derived from, isolated from, based on or homologous to the parent molecule. The term variant can be used to describe either polynucleotides or polypeptides.

As applied to polynucleotides, a variant molecule can have an entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also include polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants include nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

As applied to proteins, a variant polypeptide can have an entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also include polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants include polypeptides that contain minor, trivial, or inconsequential changes to the parent amino acid sequence. For example, minor, trivial, or inconsequential changes include amino acid changes (including substitutions, deletions and insertions) that have little or no impact on the biological activity of the polypeptide, and yield functionally identical polypeptides, including additions of non-functional peptide sequence. In other aspects, the variant polypeptides of the invention change the biological activity of the parent molecule. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made. Advantageous embodiments can involve engineered or non-naturally occurring Cas proteins having both an RNAse and DNase activity, e.g., Cas12a, or an ortholog or homolog thereof.

The term “isolated” when referring to nucleic acid molecules or polypeptides means that the nucleic acid molecule or the polypeptide is substantially free from at least one other component with which it is associated or found together in nature.

As used herein, the term “guide RNA,” “gRNA” or “sgRNA” generally refers to an RNA molecule (or a group of RNA molecules collectively) that can bind to a CRISPR protein and target the CRISPR protein to a specific location within a target DNA. A guide RNA can comprise two segments: a DNA-targeting guide segment (e.g., crRNA) and a protein-binding segment (e.g., tracrRNA). The DNA-targeting segment comprises a nucleotide sequence that is complementary to (or at least can hybridize to under stringent conditions) a target sequence.

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to or modify using a CRISPR system. A target sequence may be within a nucleic acid in vitro or in vivo within the genome of a cell, which may be any form of single-stranded or double-stranded nucleic acid.

A “target nucleic acid strand” refers to a strand of a target nucleic acid that is subject to base-pairing with a crRNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and, thus, are still capable of nucleobase complementarity.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases. As used herein, “specifically hybridizes” means the ability of an oligomeric compound to hybridize to one nucleic acid site with greater affinity than it hybridizes to another nucleic acid site. In certain embodiments, an antisense oligonucleotide specifically hybridizes to more than one target site.

As used herein, “treatment,” “treating,” “palliating,” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The terms “prevent,” “preventing,” “prevention,” “prophylactic treatment” and the like refer to reducing the probability of developing a disorder or condition (e.g., SARS-CoV-2 infection) in a subject, who does not have, but is at risk of or susceptible to developing a disorder or condition. The term includes prevention of spread of infection in a subject exposed to the virus or at risk of having SARS-CoV-2 infection.

As used herein, the term “contacting,” when used in reference to any set of components, includes any process whereby the components to be contacted are mixed into same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components, such as an Cas protein or guide RNA (or crRNA), includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

The term “mixture,” as used herein, refers to a combination of elements, that are interspersed and not in any particular order. A mixture is heterogeneous and not spatially separable into its different constituents. Examples of mixtures of elements include a number of different elements that are dissolved in the same aqueous solution or a number of different elements attached to a solid support at random or in no particular order in which the different elements are not spatially distinct. In other words, a mixture is not addressable.

The term “progeny,” such as the progeny of a transgenic plant, is one that is born of, begotten by, or derived from a plant or the transgenic plant. The introduced nucleic acid molecule may also be transiently introduced into the recipient cell such that the introduced nucleic acid molecule is not inherited by subsequent progeny and thus not considered “transgenic.” Accordingly, as used herein, a “non-transgenic” plant or plant cell is a plant which does not contain a foreign nucleic acid stably integrated into its genome.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, the term “modulate” is meant to refer to any change in biological state, i.e., increasing, decreasing, and the like.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “decrease,” “reduced,” “reduction,” “decrease,” or “inhibit” are all used herein generally to mean a decrease by a statistically significant amount. However, for avoidance of doubt, “reduced,” “reduction,” “decrease,” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

“Sample,” “test sample,” and “patient sample” may be used interchangeably herein. The sample can be a sample of serum, urine plasma, amniotic fluid, cerebrospinal fluid, cells, or tissue.

Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample,” as used herein, generally refer to a biological material being tested for and/or suspected of containing an analyte of interest such as antibodies. The sample may be any tissue sample from the subject. The sample may comprise protein from the subject.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

As used herein, the term “agent” denotes a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample,” as used herein, generally refer to a biological material being tested for and/or suspected of containing an analyte of interest such as antibodies. The sample may be any tissue sample from the subject. The sample may comprise protein from the subject.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders, and/or who has been diagnosed with inflammatory disorders. In some embodiments, the subject is a mammal. In some embodiments, the subject is human.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of the invention.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### A. EXAMPLES

### Example 1

This example describes the materials and methods used in the subsequent EXAMPLES below.

### Plasmids

gRNA was designed sequence from the MeCP2 genome using the CRISPRgRNA software program (https://www.dna20.com). The designed specific MeCP2 targeting gRNA sequence (AUGAUGGAGCGCCGCUGUUU) (SEQ ID NO: 1) was the best target sequence with significantly fewer off-targets. An empty sequence of gRNA was used as a control. Two plasmid systems were constructed to edit the mutated MeCP2 genome: (i) a combined Cas9 system with gRNA labeled pCas9-MeCP2-gRNA-Puro and (ii) a donor sequence with the GFP gene named pDonor-MeCP2-EGFP. The plasmid backbones of gRNA and pCRISPR-Cas9 were purchased from Addgene (Cambridge, Massachusetts) to construct pCas9-MeCP2-gRNA-Puro. The pCas9 plasmid was updated using a bicistronic T2A gene to express both CRISPR-Cas9 and the puromycin resistance gene cloned fused at the end of Cas9 gene using restriction enzyme PstI and PmeI: pCRISPR-Cas9-2A. The insert of U6 promoter-MeCP2gRNA was then cloned into pCRISPR-Cas9-2A using MluI and SpeI restriction enzymes as the final plasmid construction of the pCas9-MeCP2-gRNA-Puro. The pDNA4/TO plasmid was used as the backbone of the pDonorGFP plasmid. The designed donor sequences were 700-base-pair up and down-stream from the double-strand cleavage site by Cas9 and guide RNA complex. The first step is to clone the insert of CAG-GFP into the backbone plasmid using the restriction enzymes, SpeI and HindIII. Next is to clone 700-base-pair 3′ downstream side of the donor sequences from the total of 1400 base pairs using MfeI and SpeI for the vector to open the plasmid and insert EcoRI and SpeI, which EcoRI is a compatible enzyme of MfeI and SpeI. Then the next step was cloned 5′ upstream part of the donor MeCP2 gene sequence, modified 10 base pairs different from gRNA target sequence at the mutated site using compatible gene sequence for the wild-type sequence to protect donor plasmid from the MeCP2gRNA targeting: named pDonor-MeCP2-EGFP (pDonor). pCas9-Cont-gRNA-Puro was an empty control plasmid, which was created without a gRNA sequence. To improve the HDR efficiency and to prevent the genomic DNA contamination during the next-generation sequencing process, pDonor was linearized with BlpI by cutting 85 base pairs upstream from the Cas9 cut site. All plasmids were verified by sequencing before the genes were delivered to Q83X-NSCs.

### Cells

The generated control (wild type, WT) and RTT (MeCP2, Q83X) iPSC clonal lines from skin fibroblasts were differentiated as previously described. Prior to the differentiation of the iPSC clonal lines into NPCs, cells were re-plated at 30,000-40,000 cells per square cm in N2/B27 medium without FGF2 and supplemented with 5 μM Y-27632 (Stemgent, Cambridge, MA) and 1 μM retinoic acid (Tocris, Bristol, United Kingdom). Y-27632 was withdrawn on day 3 after the plating of cells, and retinoic acid was withdrawn on day 7. Starting on day 3, the medium was supplemented with 200 μM ascorbic acid (Sigma-Aldrich, St. Louis, MO), 1 μM dibutyryl-cAMP (Sigma-Aldrich, St. Louis, MO), 20 ng/ml BDNF (Life Technologies, Carlsbad, CA), and 20 ng/ml GDNF (Life Technologies, Carlsbad, CA) until day 10, after which basal NPC medium without FGF2 was used. The medium was partially changed every other day until day 21 or day 35 for further experiments. Both the Q83X-NPCs and wild-type NSCs were expanded for 3-5 passages in neural precursor medium (NPM) consisting of a 1:1 ratio of DMEM/F12 and Neurobasal medium containing 20 ng/ml FGF2 and 0.5% B27 and N2. When the cells reached 90-100% confluency, they were passaged at a ratio of 1:2 on Matrigel using Accutase. The medium was changed every other day. The experimental protocol was approved by the Biosafety Committee of Rutgers (12-325).

To induce neuronal differentiation, fresh neuronal differentiation medium (NDM, NeuroBasal medium, 1% B27, 10 ng/ml BDNF) was added. The NDM was changed every three days. The gene expression was analyzed at Day 21 through qPCR and immunocytochemistry.

### Western Blot

10×10⁶ cells were lysed on ice in RIPA buffer (20 mM Tris-HCl (pH 7.5), 150 mM NaCl 1, mM EDTA, 1% NP-40, 0.1% SDS, 0.1% sodium deoxycholate) for 20-30 min in the presence of protease inhibitors (ROCHE). The whole-cell lysates were centrifuged for 10 min at 14,000 rpm. The supernatants were transferred into new tubes, and protein concentrations were determined using the BCA protein assay (BIO-RAD). The lysates were boiled at 100° C. for 5 min and loaded on SDS-PAGE gels. Blots were probed with anti-MeCP2 (sc-137070, SANTA CRUZ BIOTECHNOLOGY) and anti-beta-actin (AC-74, SIGMA) antibodies. Blots were developed with secondary goat anti-mouse IgG HRP (ABCAM), and bands were visualized using the ECL detection kit (GE HEALTHCARE).

### Example 2

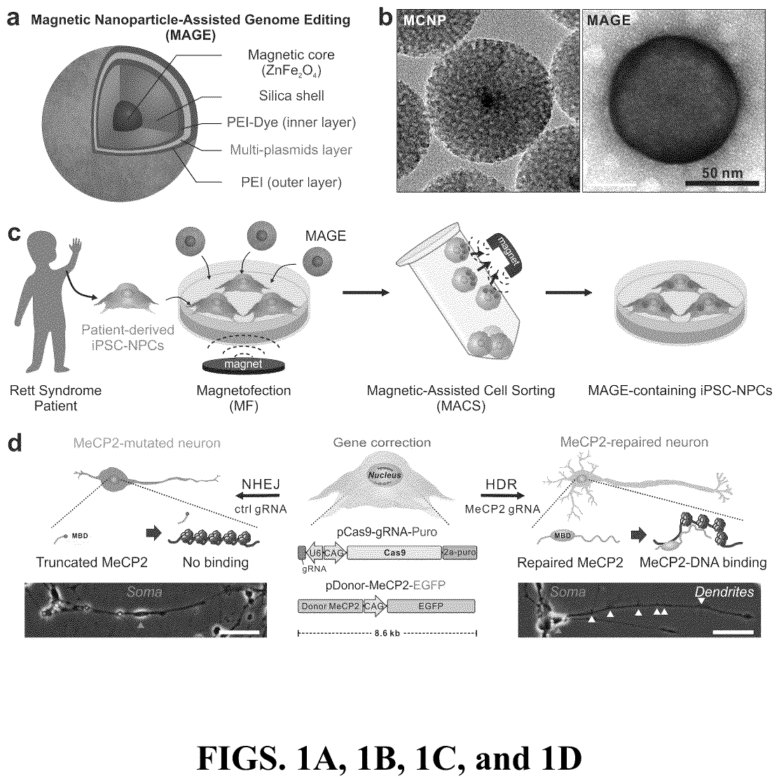

In an effort to address the low delivery efficiencies of the existing delivery methods, the present disclosure provides a novel magnetic nanoparticle-assisted genome editing (MAGE) platform, using a magnetic core-shell nanoparticle (MCNP) to deliver multiple plasmids encoding the CRISPR-Cas9 system with a magnetic force (FIGS. 1A and 1B). The underlying concept is that magnetofection first enables improved multi-plasmid cell delivery, and plasmids-containing cells can then be purified by magnetic-assisted cell sorting (MACS) (FIG. 1C). This results in the high expression of Cas9 protein and gRNA as well as the successful editing of the target mutant gene via homology-directed gene repair with the correct donor DNA sequence.

As a model system, the MAGE platform was applied to edit a mutated gene in stem cells derived from a patient with Rett syndrome, which is a non-curable rare genetic disorder caused by de novo mutations in the mutated X-linked methyl CpG-binding protein 2 (MeCP2) gene. More specifically, to demonstrate the in vitro patient-specific treatment of Rett syndrome, we generated induced pluripotent stem cell-derived neural progenitor cells (iPSC-NPCs) obtained from a Rett syndrome patient (RTT-NPCs) and designed two plasmids to cut and replace the mutated MeCP2 gene (FIG. 1D).

This study demonstrated that MAGE could effectively edit mutated MeCP2 in induced pluripotent stem cell-derived neural progenitor cells (iPSC-NPCs) obtained from a Rett syndrome patient. Through the combination of magnetofection and magnetic-activated cell sorting, MAGE enabled us to achieve significantly higher multi-plasmid delivery (99.3%) and repairing efficiencies (42.95%) in both a non-viral and non-toxic manner. Moreover, this was achieved while avoiding the size limitation of plasmids, and with significantly shorter incubation times than for conventional transfection agents. Characterization of the resulting differentiated neurons further validates MAGE, demonstrating its potential for future clinical applications.

### Example 3

### Construction of Patient-Specific CRISPR-Cas9 Vectors Targeting the Mutated MeCP2 Gene

To design the gRNA and donor sequence for the CRISPR-Cas9-based patient-specific mutated gene repair, skin fibroblasts from a male Rett syndrome patient were obtained, whose mutated MeCP2 gene sequence was previously determined and reported (Marchetto, M. C. et al. Cell 143, 527-539 (2010)). In the case of this Rett syndrome patient, the MeCP2 gene has a nonsense mutation in amino acid residue 83, which transforms glutamine (‘C’ AG) to a premature stop codon (‘T’AG), resulting in truncation and degradation of the MeCP2 protein (Q83X). To this end, two plasmid systems were designed to edit the mutated MeCP2 genome: a combined single Cas9 gene with gRNA named pCas9-gRNA-Puro (pCas9) and a donor sequence with an EGFP reporter gene named pDonor-MeCP2-EGFP (pDonor). In particular, pCas9 is a single, combined plasmid system designed to express both Cas9 double-stranded nuclease and a gRNA that was designed to target the mutated site of the MeCP2 gene (MeCP2-gRNA) as well as a selectable marker puromycin. As a control, a gRNA (control-gRNA) was constructed using an empty sequence of the target gene. Finally, to prevent the re-cleavage of repaired MeCP2 by MeCP2-gRNA and Cas9 after successful editing, a 10-base pair donor sequence was replaced with a complementary nucleic acid sequence that would not be recognized by the MeCP2-gRNA and would still translate into the correct MeCP2 amino acid sequence.

### Example 4

### Development of the MAGE Platform Using Functional Nanoparticles

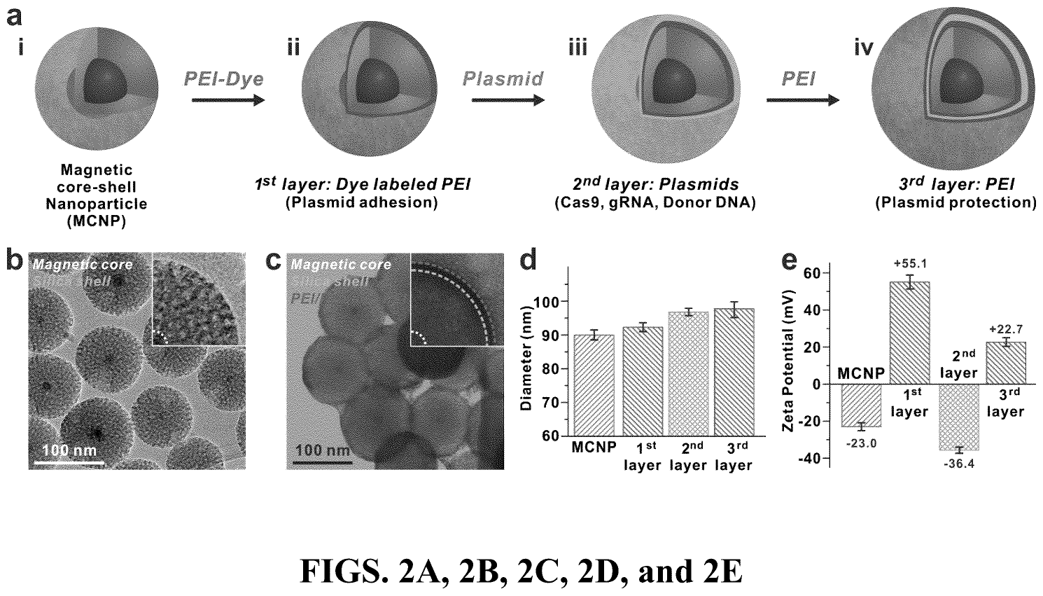

For the dual purposes of delivering plasmids encoding the CRISPR system to the iPSC-NPCs as well as tracking the nanoparticle-containing cells, MAGE was developed using layer-by-layer assembly (FIGS. 1A and 2A). The magnetic properties of the MAGE platform are derived from magnetic core-shell nanoparticles (MCNPs) with zinc-doped iron oxide (ZnFe₂O₄) cores.

This core has a significantly higher saturation magnetization when compared to conventional Fe₂O₃ or Fe₃O₄ magnetic nanoparticles (MNPs). ZnFe₂O₄ cores were first synthesized via the thermal decomposition of a mixture of metal precursors (zinc chloride, ferrous chloride, and ferric acetylacetonate) in the presence of oleic acid and oleylamine using a modified protocol (Yin, P. T. et al. Biomaterials 81, 46-57 (2016)). Following core synthesis, an inert silica shell was formed via the condensation of tetraethyl orthosilicate in the presence of a cetrimonium bromide micelle template to improve the colloidal stability and the plasmid loading capacity (Kim, J. et al. Angew Chem Int Ed Engl 47, 8438-8441 (2008)). Transmission electron microscopy (TEM) revealed that the diameter of the cores was 7.93±1.6 nm and that the MNP cores were uniformly coated with a 25.21±3.8 nm thick silica shell (FIG. 2B). For more detailed characterization, TEM revealed the monocrystalline structure of the MNP cores with a lattice fringe that was measured to be 4.8 A, which is characteristic of the (111) plane of the spinel (Shah, B., et al. Angew Chem Int Ed Engl 52, 6190-6195 (2013).). As a result, the overall diameter of the as-synthesized MCNPs was 90.12±1.37 nm.

To utilize the aforementioned MCNPs for plasmid delivery, the MCNPs were coated with two layers of branched polyethyleneimine (PEI) via electrostatic interactions in the presence of NaCl to afford the MCNPs with an overall positive charge. An initial layer of tetramethylrhodamine (TRITC)-labeled PEI was coated on the MCNP surface to allow for complexing with plasmid via electrostatic interactions as well as in-situ monitoring of plasmid release from the MCNP surface. After loading the plasmid as a second layer, an additional layer of PEI was applied to further protect the plasmid and to improve the intracellular delivery/releasing efficiency. As a result, this would facilitate MCNP complexation with plasmid DNA and induce endosomolysis within the cytoplasm (Liu, Y. et al. Anal Chem 79, 2221-2229 (2007)). The resulting MAGE platform had a final hydrodynamic size of 98.84±3.96 nm (FIGS. 2C and 2D) and zeta potential of 22.7±4.66 mV (FIG. 2E). To find the optimal concentration of plasmid-loaded MCNP-PEI while maintaining cell viability, the cytotoxic effects of different doses of particles and loaded plasmids on patient-derived RTT-NPCs were tested. To minimize cytotoxicity while maximizing transfection efficiency, 10 kDa branched PEI was used, which has previously been demonstrated to be biocompatible with stem cells. It was found that the MAGE platform was able to load more than 80% of the plasmid with a 1:15 mass ratio (plasmid:MCNP) with minimal cytotoxicity (≤70 μg/mL MCNP, 96.2% viability). Moreover, mixed populations of the pCas9 and pDonor plasmids can be loaded onto a single nanoparticle, thus allowing co-delivery of both the CRISPR-Cas9 system and the donor templates into a single cell using the MAGE platform. As such, this platform shows great potential for therapeutic genome editing owing to its ability to load multiple plasmids unlike Adeno-associated viral particles (AAV) (Zhang, Z. N. et al. Proc Natl Acad Sci 113, 3185-3190 (2016)).

### Example 5

### Magnetic-Assisted Multi-Plasmid CRISPR-Cas9 Delivery

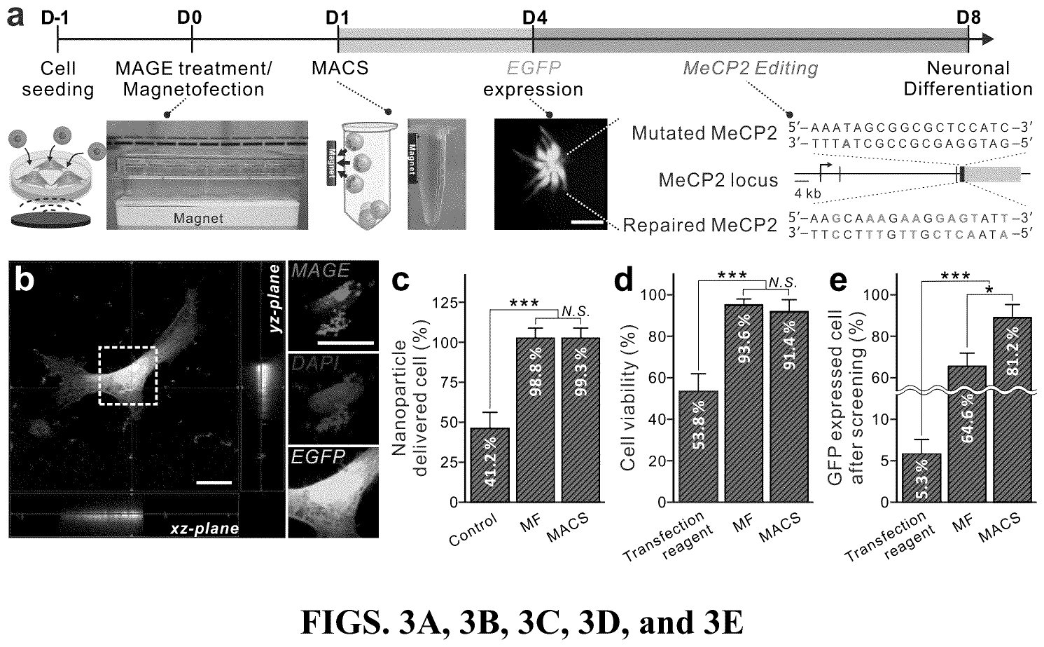

To assess the efficiency of cellular uptake of MAGE, fluorescence microscopy on iPSC-NPCs derived from the Rett syndrome patient (RTT-NPCs) and healthy donor (wild type, WT-NPCs) were performed using a previously established method (FIG. 3A) (Marchetto, M. C. et al. Cell 143, 527-539 (2010); Zhang, Z. N. et al. Proc Natl Acad Sci 113, 3185-3190 (2016)). In this case, MAGE was assembled using 2 μg of plasmids (total 2 μg, 1 μg each of pCas9 and pDonor), and delivery was enhanced using magnetofection, a well-established method that allows for the rapid accumulation of MCNPs and their payloads upon exposure to an external magnetic field. 24 h after transfection, the efficiency of uptake was visualized and quantified (FIG. 3B). It was found that the RTT-NPCs were able to efficiently uptake MAGE with magnetofection (98.8% efficiency) (FIG. 3C). Moreover, the MCNPs that remained intracellularly after magnetofection provided cellular mobility in a magnetic field, which then allowed for the application of cell sorting via MACS. MACS was able to sort out the cells that contain MAGE with 99.3% accuracy. It should be noted that, in contrast to other cationic transfection methods, which can result in low cell viabilities, MAGE and magnetofection caused little to no cytotoxicity (around 92% cell viability after MAGE delivery and magnetofection) owing to the much lower concentration and shorter time of incubation that is necessary (FIG. 3D). Specifically, it was found that most of the MAGE was secreted out from the cell via exocytosis around 48 h after the transfection, which minimized nanoparticle-mediated cytotoxicity.

Afterward, compared to commercially available transfection agents, the ratio of cells expressing EGFP due to the magnetofection of pDonor with MAGE was significantly greater. Next, in order to validate the existence of pCas9, owing to the fact that the plasmid delivered results in the expression of both Cas9 and puromycin resistance proteins, GFP expressing iPS-NPCs were screened with puromycin (FIG. 3E). The RTT-NPCs with plasmid loaded MAGEs and sorted by MACS showed a dramatically higher survival rate of GFP expressing cells with puromycin resistance. As such, the MAGE platform demonstrated its ability to effectively deliver CRISPR-Cas9 plasmid and to be used for extended incubation and downstream applications such as MeCP2 genome editing.

### Example 6

### Improving the Efficiency of Mutated MeCP2 Repair Using MAGE

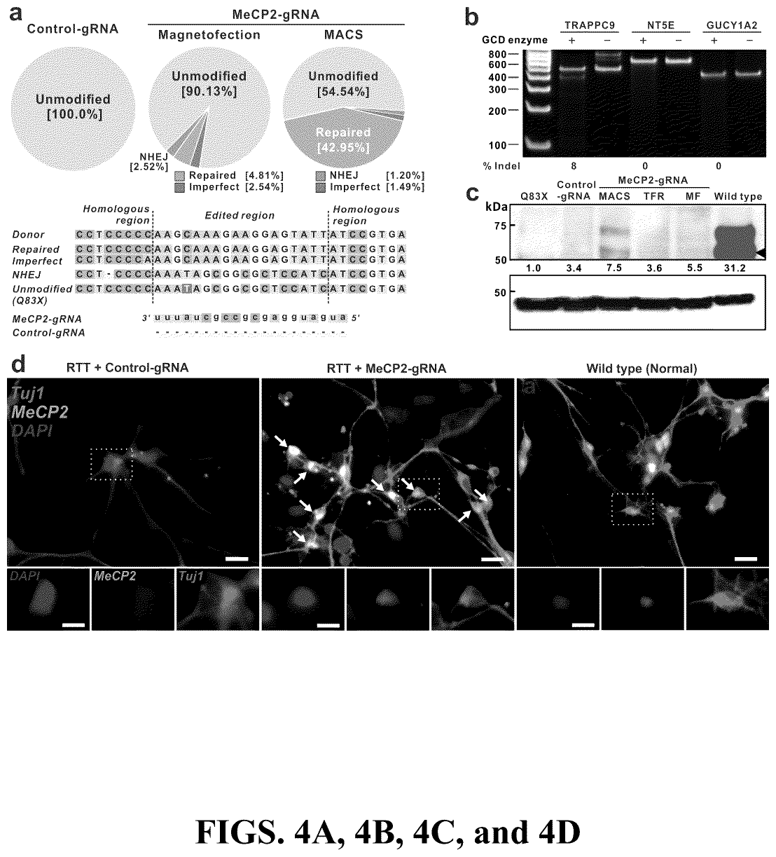

By delivering donor DNA with CRISPR-Cas9, the mutated gene can be repaired through homology-directed repair (HDR) pathway. However, the repair efficiency of HDR pathway-based therapeutic genome editing as delivered by non-viral systems have not achieved over 10%. To improve repair efficiency, two different CRISPR-Cas9 delivery methods with the MAGE platform—magnetofection and MAGE—were tested (FIG. 4). The verification of MeCP2 genome editing was accomplished by performing molecular analysis at both the DNA and protein level, including sequencing, restriction enzyme analysis, western blotting, immunostaining, and downstream gene expression profiling. First, the genome repairing efficiency was calculated based on the next-generation sequencing results of RTT-NPCs with MAGE (FIG. 4A). Compared with magnetofection, MACS showed a significant increase in HDR efficiency (42.95%) with minimal imperfections (1.49%), while magnetofection alone showed 4.81% HDR efficiency as measured using next generation sequencing analysis. The improvement in HDR efficiency resulted from the increase in intracellularly released pDonor, owing to the sorting for MAGE containing cells.

On the other hand, the high content of Cas9 plasmid in the cell has the potential to induce a higher off-target effect due to the overexpression of Cas9. Using Cas-OFFinder, an off-target prediction website, 3 potential off-target sites were identified with a parameter tolerant to a 3-bp mismatch. However, as shown in FIG. 4B, the gene cleavage detection (GCD) assay showed no significant editing occurred in these sites after HDR with MAGE.

Next, western blot was used to compare MeCP2 expression levels among plasmid-treated neurons differentiated from different NPCs (WT, RTT, edited RTT with control-gRNA, and MeCP2-gRNA) (FIG. 4C). To confirm the expression of full-length MeCP2, N-terminus targeting anti-MeCP2 antibody, which cannot attach to the truncated protein (MeCP2-Q83X), was used. Compared with RTT-neurons (non-edited), edited RTT-neurons with MeCP2-gRNA showed a significant increase in the expression of MeCP2. In particular, the expression level of MeCP2 in edited RTT-neurons was improved (36.3% increase) via the MACS process, whereas no difference was observed in control-gRNA treated RTT-neurons. Furthermore, immune-fluorescence images of the neurons showed that repaired MeCP2 was expressed and localized to the nuclei of edited RTT-neurons (47.3%), similar to WT-neurons, whereas no signal was detected in the nuclei of non-edited RTT-neurons (FIG. 4D). These results further support that the mutated MeCP2 gene was successfully replaced with donor and express the full sequence of MeCP2 in RTT-neurons that interact with organelles as normal.

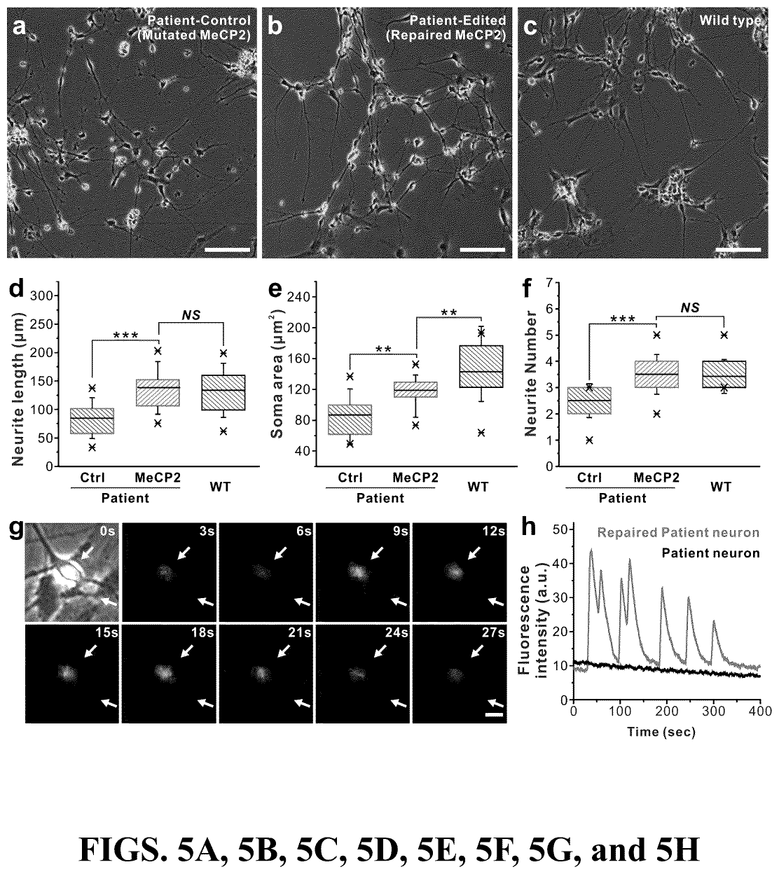

In order to validate whether the observed changes in downstream gene expression was a result of ‘repaired’ MeCP2 expression, we analyzed the resulting phenotype of the RTT-neurons before and after genome editing. A fundamental morphological phenotype that is typically seen in RTT is reduced complexity of the neuronal dendritic tree, which negatively impacts neural network development. To assay for neuronal functional recovery following editing of the mutated MeCP2 gene, the edited RTT cells were differentiated into neurons using neuronal differentiation medium and compared with wild-type neurons. As expected, the WT-neuron and ‘edited’ RTT-neuron showed significantly different phenotypes when compared with ‘mutated’ RTT-neuron, including the length of neurites, soma size, and the number of neurites per cell (FIG. 5). To compare the ‘edited’ and ‘mutated’ effects of RTT-neurons, MeCP2-gRNA, and control-gRNA treated RTT-NPCs were differentiated. The WT-neuron and ‘edited’ RTT-neurons have a similar morphology (FIGS. 5A, 5B, and 5C), whereas there was a significant difference in neurite length when comparing with ‘mutated’ RTT-neurons (FIG. 5D). In particular, the ‘edited’ RTT-neurons showed up to 76.2% higher neurite outgrowth when compared with ‘mutated’ neurons, and this result correlated well with BDNF and Reelin expression levels, which acts as the signal for proper axonal growth. In addition, the size of the soma and the number of neurites in the ‘edited’ RTT-neurons increased by up to 22.8% and 16.7%, respectively (FIGS. 5E and 5F). In particular, ‘edited’ RTT-neurons showed improved dendritic growth of neurites, which was accompanied by a decrease in FXYD1 and DLX5 expression.

Furthermore, the functional recovery of RTT-neurons that were edited using the MAGE platform was confirmed. To this end, changes in intracellular calcium levels of treated and untreated cells were monitored, as functionally active neurons should spontaneously fire action potentials that allow for the influx of cations, including calcium. To accomplish this, a commercially available calcium indicator dye, Fluo 4AM, was used to monitor changes in intracellular calcium concentrations via the visualization and quantification of fluorescence intensity. 21 days post-differentiation, calcium imaging was performed. A representative example of calcium tracing in ‘edited’ and ‘mutated’ RTT-neurons is depicted in FIG. 5G. The frequency of calcium oscillations in ‘edited’ RTT-neurons expressing MeCP2 was greater when compared to ‘mutated’ RTT, suggesting a recovery in neuronal network connectivity and activity dynamics (FIG. 5H). These results indicated that the edited RTT-neurons show functional activity.

### DISCUSSION

In this work, a MAGE platform was developed that significantly improves the genome editing efficiency of CRISPR-Cas9. This MAGE platform was composed of a MCNP with zinc-doped iron oxide (ZnFe₂O₄) core and inert mesoporous silica shells, which were coated with two layers of branched PEI to allow for the delivery of multiple plasmids via electrostatic interaction. Plasmids were linearized by restriction enzymes prior to loading on the MAGE platform to prevent uncontrolled amplification of delivered plasmids as uncontrolled amplification of exogenous Cas9 plasmids increases the chance of off-target editing, which is an intrinsic limitation of Adeno-associated viral (AAV) particles. Furthermore, using a linearized donor template increases the HDR efficiency by creating sticky ends that facilitate the hybridization between the homology regions of the donor DNA and its target.

As a proof-of-concept, this disclosure demonstrated the effective application of the MAGE platform for the editing of the mutated MeCP2 gene in iPSC-NPCs obtained from a Rett syndrome patient. In particular, this MAGE platform holds a number of key advantages, including (i) faster and more efficient delivery of multiple genes due to magnetofection, (ii) magnetic-activated cell sorting (MACS), iii) enhanced biocompatibility, and (iv) in-situ, real-time tracking of the delivery process, which all synergistically enhance the gene correction efficiency of CRISPR-Cas9.

The patient-derived cells showed dramatic phenotypic improvement after MeCP2 repair. Even though there was not 100% editing efficiency, mutated cells showed distinctive phenotypic improvements when they were grown with the repaired cells, owing to secreted exosomes from the repaired cells. Therefore, there is the potential for functional improvement of genomic disorder patients following ex vivo CRISPR treatment with MAGE, in which autologous cells are taken from the patient, edited in the laboratory, and re-introduced into the patient, even without replacing all mutated cells. As such, we believe it holds great promise for future clinical applications.

In conclusion, this disclosure successfully demonstrated the ability of the disclosed multifunctional MAGE platform to efficiently deliver CRISPR-Cas9 to stem cells. Owing to the great potential of CRISPR-Cas9, the implications of this study go well beyond the treatment of Rett syndrome and can potentially be used for a host of applications in stem cell therapy for genetic disorders.

### Representative Sequences

| SEQ ID | | |
|---|---|---|
| NO | SEQUENCE | NOTES |
| 2 | cccagtacatgaccttatgggactttcctacttggcagtacatctacgtattagtcatcgctattaccatggtg | pCas9- |
| | atgcggttttggcagtacatcaatgggcgtggatagcggtttgactcacggggatttccaagtctccaccc | MeCP2- |
| | cattgacgtcaatgggagtttgttttggcaccaaaatcaacgggactttccaaaatgtcgtaacaactccgc | gRNA- |
| | cccattgacgcaaatgggcggtaggcgtgtacggtgggaggtctatataagcagagctggtttagtgaac | Puro |
| | cgtcagatccgctagagatccgcggccgctaatacgactcactatagggagagccgccaccatggataa | |
| | aaagtattctattggtttagacatcggcactaattccgttggatgggctgtcataaccgatgaatacaaagta | |
| | ccttcaaagaaatttaaggtgttggggaacacagaccgtcattcgattaaaaagaatcttatcggtgccctc | |
| | ctattcgatagtggcgaaacggcagaggcgactcgcctgaaacgaaccgctcggagaaggtatacacg | |
| | tcgcaagaaccgaatatgttacttacaagaaatttttagcaatgagatggccaaagttgacgattctttctttc | |
| | accgtttggaagagtccttccttgtcgaagaggacaagaaacatgaacggcaccccatctttggaaacat | |
| | agtagatgaggtggcatatcatgaaaagtacccaacgatttatcacctcagaaaaaagctagttgactcaa | |
| | ctgataaagcggacctgaggttaatctacttggctcttgcccatatgataaagttccgtgggcactttctcatt | |
| | gagggtgatctaaatccggacaactcggatgtcgacaaactgttcatccagttagtacaaacctataatca | |
| | gttgtttgaagagaaccctataaatgcaagtggcgtggatgcgaaggctattcttagcgcccgcctctcta | |
| | aatcccgacggctagaaaacctgatcgcacaattacccggagagaagaaaaatgggttgttcggtaacc | |
| | ttatagcgctctcactaggcctgacaccaaattttaagtcgaacttcgacttagctgaagatgccaaattgca | |
| | gcttagtaaggacacgtacgatgacgatctcgacaatctactggcacaaattggagatcagtatgcggac | |
| | ttatttttggctgccaaaaaccttagcgatgcaatcctcctatctgacatactgagagttaatactgagattac | |
| | caaggcgccgttatccgcttcaatgatcaaaaggtacgatgaacatcaccaagacttgacacttctcaagg | |
| | ccctagtccgtcagcaactgcctgagaaatataaggaaatattctttgatcagtcgaaaaacgggtacgca | |
| | ggttatattgacggcggagcgagtcaagaggaattctacaagtttatcaaacccatattagagaagatgga | |
| | tgggacggaagagttgcttgtaaaactcaatcgcgaagatctactgcgaaagcagcggactttcgacaac | |
| | ggtagcattccacatcaaatccacttaggcgaattgcatgctatacttagaaggcaggaggatttttatccgt | |
| | tcctcaaagacaatcgtgaaaagattgagaaaatcctaacctttcgcataccttactatgtgggacccctgg | |
| | cccgagggaactctcggttcgcatggatgacaagaaagtccgaagaaacgattactccatggaattttga | |
| | ggaagttgtcgataaaggtgcgtcagctcaatcgttcatcgagaggatgaccaactttgacaagaatttac | |
| | cgaacgaaaaagtattgcctaagcacagtttactttacgagtatttcacagtgtacaatgaactcacgaaag | |
| | ttaagtatgtcactgagggcatgcgtaaacccgcctttctaagcggagaacagaagaaagcaatagtaga | |
| | tctgttattcaagaccaaccgcaaagtgacagttaagcaattgaaagaggactactttaagaaaattgaatg | |
| | cttcgattctgtcgagatctccggggtagaagatcgatttaatgcgtcacttggtacgtatcatgacctccta | |
| | aagataattaaagataaggacttcctggataacgaagagaatgaagatatcttagaagatatagtgttgact | |
| | cttaccctctttgaagatcgggaaatgattgaggaaagactaaaaacatacgctcacctgttcgacgataa | |
| | ggttatgaaacagttaaagaggcgtcgctatacgggctggggacgattgtcgcggaaacttatcaacgg | |
| | gataagagacaagcaaagtggtaaaactattctcgattttctaaagagcgacggcttcgccaataggaact | |
| | ttatgcagctgatccatgatgactctttaaccttcaaagaggatatacaaaaggcacaggtttccggacaag | |
| | gggactcattgcacgaacatattgcgaatcttgctggttcgccagccatcaaaaagggcatactccagac | |
| | agtcaaagtagtggatgagctagttaaggtcatgggacgtcacaaaccggaaaacattgtaatcgagatg | |
| | gcacgcgaaaatcaaacgactcagaaggggcaaaaaaacagtcgagagcggatgaagagaatagaa | |
| | gagggtattaaagaactgggcagccagatcttaaaggagcatcctgtggaaaatacccaattgcagaac | |
| | gagaaactttacctctattacctacaaaatggaagggacatgtatgttgatcaggaactggacataaaccgt | |
| | ttatctgattacgacgtcgatcacattgtaccccaatcctttttgaaggacgattcaatcgacaataaagtgct | |
| | tacacgctcggataagaaccgagggaaaagtgacaatgttccaagcgaggaagtcgtaaagaaaatga | |
| | agaactattggcggcagctcctaaatgcgaaactgataacgcaaagaaagttcgataacttaactaaagct | |
| | gagaggggtggcttgtctgaacttgacaaggccggatttattaaacgtcagctcgtggaaacccgccaaa | |
| | tcacaaagcatgttgcacagatactagattcccgaatgaatacgaaatacgacgagaacgataagctgatt | |
| | cgggaagtcaaagtaatcactttaaagtcaaaattggtgtcggacttcagaaaggattttcaattctataaag | |
| | ttagggagataaataactaccaccatgcgcacgacgcttatcttaatgccgtcgtagggaccgcactcatt | |
| | aagaaatacccgaagctagaaagtgagtttgtgtatggtgattacaaagtttatgacgtccgtaagatgatc | |
| | gcgaaaagcgaacaggagataggcaaggctacagccaaatacttcttttattctaacattatgaatttcttta | |
| | agacggaaatcactctggcaaacggagagatacgcaaacgacctttaattgaaaccaatggggagaca | |
| | ggtgaaatcgtatgggataagggccgggacttcgcgacggtgagaaaagttttgtccatgccccaagtc | |
| | aacatagtaaagaaaactgaggtgcagaccggagggttttcaaaggaatcgattcttccaaaaaggaata | |
| | gtgataagctcatcgctcgtaaaaaggactgggacccgaaaaagtacggtggcttcgatagccctacagt | |
| | tgcctattctgtcctagtagtggcaaaagttgagaagggaaaatccaagaaactgaagtcagtcaaagaat | |
| | tattggggataacgattatggagcgctcgtcttttgaaaagaaccccatcgacttccttgaggcgaaaggtt | |
| | acaaggaagtaaaaaaggatctcataattaaactaccaaagtatagtctgtttgagttagaaaatggccga | |
| | aaacggatgttggctagcgccggagagcttcaaaaggggaacgaactcgcactaccgtctaaatacgtg | |
| | aatttcctgtatttagcgtcccattacgagaagttgaaaggttcacctgaagataacgaacagaagcaactt | |
| | tttgttgagcagcacaaacattatctcgacgaaatcatagagcaaatttcggaattcagtaagagagtcatc | |
| | ctagctgatgccaatctggacaaagtattaagcgcatacaacaagcacagggataaacccatacgtgag | |
| | caggcggaaaatattatccatttgtttactcttaccaacctcggcgctccagccgcattcaagtattttgaca | |
| | caacgatagatcgcaaacgatacacttctaccaaggaggtgctagacgcgacactgattcaccaatccat | |
| | cacgggattatatgaaactcggatagatttgtcacagcttgggggtgacggatcccccaagaagaagag | |
| | gaaagtctcgagcgactacaaagaccatgacggtgattataaagatcatgacatcgattacaaggatgac | |
| | gatgacaaggctgcagacgagggcagaggaagtcttctaacatgcggtgacgtggaggagaatcccg | |
| | gccctatgaccgagtacaagcccacggtgcgcctcgccacccgcgacgacgtccccagggccgtacg | |
| | caccctcgccgccgcgttcgccgactaccccgccacgcgccacaccgtcgatccggaccgccacatc | |
| | gagcgggtcaccgagctgcaagaactcttcctcacgcgcgtcgggctcgacatcggcaaggtgtgggt | |
| | cgcggacgacggcgccgcggtggcggtctggaccacgccggagagcgtcgaagcgggggcggtgt | |
| | tcgccgagatcggcccgcgcatggccgagttgagcggttcccggctggccgcgcagcaacagatgga | |
| | aggcctcctggcgccgcaccggcccaaggagcccgcgtggttcctggccaccgtcggcgtctcgccc | |
| | gaccaccagggcaagggtctgggcagcgccgtcgtgctccccggagtggaggcggccgagcgcgc | |
| | cggggtgcccgccttcctggagacctccgcgccccgcaacctccccttctacgagcggctcggcttcac | |
| | cgtcaccgccgacgtcgaggtgcccgaaggaccgcgcacctggtgcatgacccgcaagcccggtgcc | |
| | tgacaattggatatccccgggaaaaacccgctgatcagcctcgactgtgccttctagttgccagccatctg | |
| | ttgtttgcccctcccccgtgccttccttgaccctggaaggtgccactcccactgtcctttcctaataaaatga | |
| | ggaaattgcatcgcattgtctgagtaggtgtcattctattctggggggtggggggggcaggacagcaag | |
| | ggggaggattgggaagacaatagcaggcatgctggggatgcggtgggctctatggcttctgaggcgga | |
| | aagaaccagctggggctcgataccgtcgacctctagctagagcttggcgtaatcatggtcatagctgtttc | |
| | ctgtgtgaaattgttatccgctcacaattccacacaacatacgagccggaagcataaagtgtaaagcctag | |
| | ggtgcctaatgagtgagctaactcacattaattgcgttgcgctcactgcccgctttccagtcgggaaacctg | |
| | tcgtgccagctgcattaatgaatcggccaacgcgcggggagaggcggtttgcgtattgggcgctcttccg | |
| | cttcctcgctcactgactcgctgcgctcggtcgttcggctgcggcgagcggtatcagctcactcaaaggc | |
| | ggtaatacggttatccacagaatcaggggataacgcaggaaagaacatgtgagcaaaaggccagcaaa | |
| | aggccaggaaccgtaaaaaggccgcgttgctggcgtttttccataggctccgcccccctgacgagcatc | |
| | acaaaaatcgacgctcaagtcagaggtggcgaaacccgacaggactataaagataccaggcgtttccc | |
| | cctggaagctccctcgtgcgctctcctgttccgaccctgccgcttaccggatacctgtccgcctttctccctt | |
| | cgggaagcgtggcgctttctcaatgctcacgctgtaggtatctcagttcggtgtaggtcgttcgctccaag | |
| | ctgggctgtgtgcacgaaccccccgttcagcccgaccgctgcgccttatccggtaactatcgtcttgagtc | |
| | caacccggtaagacacgacttatcgccactggcagcagccactggtaacaggattagcagagcgaggt | |
| | atgtaggcggtgctacagagttcttgaagtggtggcctaactacggctacactagaaggacagtatttggt | |
| | atctgcgctctgctgaagccagttaccttcggaaaaagagttggtagctcttgatccggcaaacaaaccac | |
| | cgctggtagcggtggtttttttgtttgcaagcagcagattacgcgcagaaaaaaaggatctcaagaagatc | |
| | ctttgatcttttctacggggtctgacgctcagtggaacgaaaactcacgttaagggattttggtcatgagatt | |
| | atcaaaaaggatcttcacctagatccttttaaattaaaaatgaagttttaaatcaatctaaagtatatatgagta | |
| | aacttggtctgacagttaccaatgcttaatcagtgaggcacctatctcagcgatctgtctatttcgttcatcca | |
| | tagttgcctgactccccgtcgtgtagataactacgatacgggagggcttaccatctggccccagtgctgca | |
| | atgataccgcgagacccacgctcaccggctccagatttatcagcaataaaccagccagccggaagggc | |
| | cgagcgcagaagtggtcctgcaactttatccgcctccatccagtctattaattgttgccgggaagctagagt | |
| | aagtagttcgccagttaatagtttgcgcaacgttgttgccattgctacaggcatcgtggtgtcacgctcgtc | |
| | gtttggtatggcttcattcagctccggttcccaacgatcaaggcgagttacatgatcccccatgttgtgcaa | |
| | aaaagcggttagctccttcggtcctccgatcgttgtcagaagtaagttggccgcagtgttatcactcatggtt | |
| | atggcagcactgcataattctcttactgtcatgccatccgtaagatgcttttctgtgactggtgagtactcaac | |
| | caagtcattctgagaatagtgtatgcggcgaccgagttgctcttgcccggcgtcaatacgggataataccg | |
| | cgccacatagcagaactttaaaagtgctcatcattggaaaacgttcttcggggcgaaaactctcaaggatc | |
| | ttaccgctgttgagatccagttcgatgtaacccactcgtgcacccaactgatcttcagcatcttttactttcac | |
| | cagcgtttctgggtgagcaaaaacaggaaggcaaaatgccgcaaaaaagggaataagggcgacacgg | |
| | aaatgttgaatactcatactcttcctttttcaatattattgaagcatttatcagggttattgtctcatgagcggata | |
| | catatttgaatgtatttagaaaaataaacaaataggggttccgcgcacatttccccgaaaagtgccacctga | |
| | cgtcgacggatcgggagatcgatctcccgatcccctagggtcgactctcagtacaatctgctctgatgcc | |
| | gcatagttaagccagtatctgctccctgcttgtgtgttggaggtcgctgagtagtgcgcgagcaaaatttaa | |
| | gctacaacaaggcaaggcttgaccgacaattgcatgaagaatctgcttagggttaggcgttttgcgctgct | |
| | tcgcgatgtacgggccagatatactagtaaggtcgggcaggaagagggcctatttcccatgattccttcat | |
| | atttgcatatacgatacaaggctgttagagagataattagaattaatttgactgtaaacacaaagatattagta | |
| | caaaatacgtgacgtagaaagtaataatttcttgggtagtttgcagttttaaaattatgttttaaaatggactat | |
| | catatgcttaccgtaacttgaaagtatttcgatttcttggctttatatatcttgtggaaaggacgaaacaccgat | |
| | gatggagcgccgctgtttgttttagagctagaaatagcaagttaaaataaggctagtccgttatcaacttga | |
| | aaaagtggcaccgagtcggtgctttttttaagcacgcgttattaatagtaatcaattacggggtcattagttca | |
| | tagcccatatatggagttccgcgttacataacttacggtaaatggcccgcctggctgaccgcccaacgac | |
| | ccccgcccattgacgtcaataatgacgtatgttcccatagtaacgccaatagggactttccattgacgtcaa | |
| | tgggtggactatttacggtaaactgcccacttggcagtacatcaagtgtatc | |
| 3 | atatgccaagtacgccccctattgacgtcaatgacggtaaatggcccgcctggcattatgcccagtacatg | Cas9 |
| | accttatgggactttcctacttggcagtacatctacgtattagtcatcgctattaccatggtgatgcggttttgg | (Addgene |
| | cagtacatcaatgggcgtggatagcggtttgactcacggggatttccaagtctccaccccattgacgtcaa | 43861 |
| | tgggagtttgttttggcaccaaaatcaacgggactttccaaaatgtcgtaacaactccgccccattgacgca | cas9 |
| | aatgggcggtaggcgtgtacggtgggaggtctatataagcagagctggtttagtgaaccgtcagatccg | sequnece) |
| | ctagagatccgcggccgctaatacgactcactatagggagagccgccaccatggataaaaagtattctat | |
| | tggtttagacatcggcactaattccgttggatgggctgtcataaccgatgaatacaaagtaccttcaaagaa | |
| | atttaaggtgttggggaacacagaccgtcattcgattaaaaagaatcttatcggtgccctcctattcgatagt | |
| | ggcgaaacggcagaggcgactcgcctgaaacgaaccgctcggagaaggtatacacgtcgcaagaac | |
| | cgaatatgttacttacaagaaatttttagcaatgagatggccaaagttgacgattctttctttcaccgtttggaa | |
| | gagtccttccttgtcgaagaggacaagaaacatgaacggcaccccatctttggaaacatagtagatgagg | |
| | tggcatatcatgaaaagtacccaacgatttatcacctcagaaaaaagctagttgactcaactgataaagcg | |
| | gacctgaggttaatctacttggctcttgcccatatgataaagttccgtgggcactttctcattgagggtgatct | |
| | aaatccggacaactcggatgtcgacaaactgttcatccagttagtacaaacctataatcagttgtttgaaga | |
| | gaaccctataaatgcaagtggcgtggatgcgaaggctattcttagcgcccgcctctctaaatcccgacgg | |
| | ctagaaaacctgatcgcacaattacccggagagaagaaaaatgggttgttcggtaaccttatagcgctctc | |
| | actaggcctgacaccaaattttaagtcgaacttcgacttagctgaagatgccaaattgcagcttagtaagga | |
| | cacgtacgatgacgatctcgacaatctactggcacaaattggagatcagtatgcggacttatttttggctgc | |
| | caaaaaccttagcgatgcaatcctcctatctgacatactgagagttaatactgagattaccaaggcgccgtt | |
| | atccgcttcaatgatcaaaaggtacgatgaacatcaccaagacttgacacttctcaaggccctagtccgtc | |
| | agcaactgcctgagaaatataaggaaatattctttgatcagtcgaaaaacgggtacgcaggttatattgac | |
| | ggcggagcgagtcaagaggaattctacaagtttatcaaacccatattagagaagatggatgggacggaa | |
| | gagttgcttgtaaaactcaatcgcgaagatctactgcgaaagcagcggactttcgacaacggtagcattcc | |
| | acatcaaatccacttaggcgaattgcatgctatacttagaaggcaggaggatttttatccgttcctcaaaga | |
| | caatcgtgaaaagattgagaaaatcctaacctttcgcataccttactatgtgggacccctggcccgaggga | |
| | actctcggttcgcatggatgacaagaaagtccgaagaaacgattactccatggaattttgaggaagttgtc | |
| | gataaaggtgcgtcagctcaatcgttcatcgagaggatgaccaactttgacaagaatttaccgaacgaaa | |
| | aagtattgcctaagcacagtttactttacgagtatttcacagtgtacaatgaactcacgaaagttaagtatgtc | |
| | actgagggcatgcgtaaacccgcctttctaagcggagaacagaagaaagcaatagtagatctgttattca | |
| | agaccaaccgcaaagtgacagttaagcaattgaaagaggactactttaagaaaattgaatgcttcgattct | |
| | gtcgagatctccggggtagaagatcgatttaatgcgtcacttggtacgtatcatgacctcctaaagataatt | |
| | aaagataaggacttcctggataacgaagagaatgaagatatcttagaagatatagtgttgactcttaccctc | |
| | tttgaagatcgggaaatgattgaggaaagactaaaaacatacgctcacctgttcgacgataaggttatgaa | |
| | acagttaaagaggcgtcgctatacgggctggggacgattgtcgcggaaacttatcaacgggataagaga | |
| | caagcaaagtggtaaaactattctcgattttctaaagagcgacggcttcgccaataggaactttatgcagct | |
| | gatccatgatgactctttaaccttcaaagaggatatacaaaaggcacaggtttccggacaaggggactcat | |
| | tgcacgaacatattgcgaatcttgctggttcgccagccatcaaaaagggcatactccagacagtcaaagt | |
| | agtggatgagctagttaaggtcatgggacgtcacaaaccggaaaacattgtaatcgagatggcacgcga | |
| | aaatcaaacgactcagaaggggcaaaaaaacagtcgagagcggatgaagagaatagaagagggtatt | |
| | aaagaactgggcagccagatcttaaaggagcatcctgtggaaaatacccaattgcagaacgagaaacttt | |
| | acctctattacctacaaaatggaagggacatgtatgttgatcaggaactggacataaaccgtttatctgatta | |
| | cgacgtcgatcacattgtaccccaatcctttttgaaggacgattcaatcgacaataaagtgcttacacgctc | |
| | ggataagaaccgagggaaaagtgacaatgttccaagcgaggaagtcgtaaagaaaatgaagaactatt | |
| | ggcggcagctcctaaatgcgaaactgataacgcaaagaaagttcgataacttaactaaagctgagaggg | |
| | gtggcttgtctgaacttgacaaggccggatttattaaacgtcagctcgtggaaacccgccaaatcacaaag | |
| | catgttgcacagatactagattcccgaatgaatacgaaatacgacgagaacgataagctgattcgggaag | |
| | tcaaagtaatcactttaaagtcaaaattggtgtcggacttcagaaaggattttcaattctataaagttaggga | |
| | gataaataactaccaccatgcgcacgacgcttatcttaatgccgtcgtagggaccgcactcattaagaaat | |
| | acccgaagctagaaagtgagtttgtgtatggtgattacaaagtttatgacgtccgtaagatgatcgcgaaa | |
| | agcgaacaggagataggcaaggctacagccaaatacttcttttattctaacattatgaatttctttaagacgg | |
| | aaatcactctggcaaacggagagatacgcaaacgacctttaattgaaaccaatggggagacaggtgaaa | |
| | tcgtatgggataagggccgggacttcgcgacggtgagaaaagttttgtccatgccccaagtcaacatagt | |
| | aaagaaaactgaggtgcagaccggagggttttcaaaggaatcgattcttccaaaaaggaatagtgataag | |
| | ctcatcgctcgtaaaaaggactgggacccgaaaaagtacggtggcttcgatagccctacagttgcctattc | |
| | tgtcctagtagtggcaaaagttgagaagggaaaatccaagaaactgaagtcagtcaaagaattattgggg | |
| | ataacgattatggagcgctcgtcttttgaaaagaaccccatcgacttccttgaggcgaaaggttacaagga | |
| | agtaaaaaaggatctcataattaaactaccaaagtatagtctgtttgagttagaaaatggccgaaaacggat | |
| | gttggctagcgccggagagcttcaaaaggggaacgaactcgcactaccgtctaaatacgtgaatttcctg | |
| | tatttagcgtcccattacgagaagttgaaaggttcacctgaagataacgaacagaagcaactttttgttgag | |
| | cagcacaaacattatctcgacgaaatcatagagcaaatttcggaattcagtaagagagtcatcctagctga | |
| | tgccaatctggacaaagtattaagcgcatacaacaagcacagggataaacccatacgtgagcaggcgg | |
| | aaaatattatccatttgtttactcttaccaacctcggcgctccagccgcattcaagtattttgacacaacgata | |
| | gatcgcaaacgatacacttctaccaaggaggtgctagacgcgacactgattcaccaatccatcacgggat | |
| | tatatgaaactcggatagatttgtcacagcttgggggtgacggatcccccaagaagaagaggaaagtctc | |
| | gagcgactacaaagaccatgacggtgattataaagatcatgacatcgattacaaggatgacgatgacaa | |
| | ggctgcaggatgaccggtcatcatcaccatcaccattgagtttaaacccgctgatcagcctcgactgtgcc | |
| | ttctagttgccagccatctgttgtttgcccctcccccgtgccttccttgaccctggaaggtgccactcccact | |
| | gtcctttcctaataaaatgaggaaattgcatcgcattgtctgagtaggtgtcattctattctggggggtgggg | |
| | tggggcaggacagcaagggggaggattgggaagacaatagcaggcatgctggggatgcggtgggct | |
| | ctatggcttctgaggcggaaagaaccagctggggctcgataccgtcgacctctagctagagcttggcgta | |
| | atcatggtcatagctgtttcctgtgtgaaattgttatccgctcacaattccacacaacatacgagccggaag | |
| | cataaagtgtaaagcctagggtgcctaatgagtgagctaactcacattaattgcgttgcgctcactgcccg | |
| | ctttccagtcgggaaacctgtcgtgccagctgcattaatgaatcggccaacgcgcggggagaggcggtt | |
| | tgcgtattgggcgctcttccgcttcctcgctcactgactcgctgcgctcggtcgttcggctgcggcgagcg | |
| | gtatcagctcactcaaaggcggtaatacggttatccacagaatcaggggataacgcaggaaagaacatg | |
| | tgagcaaaaggccagcaaaaggccaggaaccgtaaaaaggccgcgttgctggcgtttttccataggctc | |
| | cgcccccctgacgagcatcacaaaaatcgacgctcaagtcagaggtggcgaaacccgacaggactata | |
| | aagataccaggcgtttccccctggaagctccctcgtgcgctctcctgttccgaccctgccgcttaccggat | |
| | acctgtccgcctttctcccttcgggaagcgtggcgctttctcaatgctcacgctgtaggtatctcagttcggt | |
| | gtaggtcgttcgctccaagctgggctgtgtgcacgaaccccccgttcagcccgaccgctgcgccttatcc | |
| | ggtaactatcgtcttgagtccaacccggtaagacacgacttatcgccactggcagcagccactggtaaca | |
| | ggattagcagagcgaggtatgtaggcggtgctacagagttcttgaagtggtggcctaactacggctacac | |
| | tagaaggacagtatttggtatctgcgctctgctgaagccagttaccttcggaaaaagagttggtagctcttg | |
| | atccggcaaacaaaccaccgctggtagcggtggtttttttgtttgcaagcagcagattacgcgcagaaaa | |
| | aaaggatctcaagaagatcctttgatcttttctacggggtctgacgctcagtggaacgaaaactcacgttaa | |
| | gggattttggtcatgagattatcaaaaaggatcttcacctagatccttttaaattaaaaatgaagttttaaatca | |
| | atctaaagtatatatgagtaaacttggtctgacagttaccaatgcttaatcagtgaggcacctatctcagcga | |
| | tctgtctatttcgttcatccatagttgcctgactccccgtcgtgtagataactacgatacgggagggcttacc | |
| | atctggccccagtgctgcaatgataccgcgagacccacgctcaccggctccagatttatcagcaataaac | |
| | cagccagccggaagggccgagcgcagaagtggtcctgcaactttatccgcctccatccagtctattaatt | |
| | gttgccgggaagctagagtaagtagttcgccagttaatagtttgcgcaacgttgttgccattgctacaggca | |
| | tcgtggtgtcacgctcgtcgtttggtatggcttcattcagctccggttcccaacgatcaaggcgagttacat | |
| | gatcccccatgttgtgcaaaaaagcggttagctccttcggtcctccgatcgttgtcagaagtaagttggcc | |
| | gcagtgttatcactcatggttatggcagcactgcataattctcttactgtcatgccatccgtaagatgcttttct | |
| | gtgactggtgagtactcaaccaagtcattctgagaatagtgtatgcggcgaccgagttgctcttgcccggc | |
| | gtcaatacgggataataccgcgccacatagcagaactttaaaagtgctcatcattggaaaacgttcttcgg | |
| | ggcgaaaactctcaaggatcttaccgctgttgagatccagttcgatgtaacccactcgtgcacccaactga | |
| | tcttcagcatcttttactttcaccagcgtttctgggtgagcaaaaacaggaaggcaaaatgccgcaaaaaa | |
| | gggaataagggcgacacggaaatgttgaatactcatactcttcctttttcaatattattgaagcatttatcagg | |
| | gttattgtctcatgagcggatacatatttgaatgtatttagaaaaataaacaaataggggttccgcgcacattt | |
| | ccccgaaaagtgccacctgacgtcgacggatcgggagatcgatctcccgatcccctagggtcgactctc | |
| | agtacaatctgctctgatgccgcatagttaagccagtatctgctccctgcttgtgtgttggaggtcgctgagt | |
| | agtgcgcgagcaaaatttaagctacaacaaggcaaggcttgaccgacaattgcatgaagaatctgcttag | |
| | ggttaggcgttttgcgctgcttcgcgatgtacgggccagatatacgcgttgacattgattattgactagttatt | |
| | aatagtaatcaattacggggtcattagttcatagcccatatatggagttccgcgttacataacttacggtaaat | |
| | ggcccgcctggctgaccgcccaacgacccccgcccattgacgtcaataatgacgtatgttcccatagtaa | |
| | cgccaatagggactttccattgacgtcaatgggggactatttacggtaaactgcccacttggcagtacatc | |
| | aagtgtatc | |
| 4 | acggatcgggagatctcccgatcccctatggtgcactctcagtacaatctgctctgatgccgcatagttaa | pMeCP2 |
| | gccagtatctgctccctgcttgtgtgttggaggtcgctgagtagtgcgcgagcaaaatttaagctacaaca | (donor)- |
| | aggcaaggcttgaccgacaattccaaagccactcactcgtctgcctgccgctgacccccccaggcctct | GFP |
| | ccaaagttcagcaaccaaagagtcaggcctggtacagaagggttgtcaggctgaggcccattctgctgg | |
| | ttggtgcctgggcctgagtcaccatctagaacattcccaagtttgaactcaggtacggtgctcagtctctcc | |
| | aggaatcagtttcaacaagataatcctttgctcacccttctcatggaattaaatgaggctgtctgcaaatgtc | |
| | atttgaaaactgctaacctttttgggatcatttaaaaggcagaactgaaacatgcttcttcacccctctgaacc | |
| | cctggctctgcaagttcctctgcttccgcagctattccatccccagataaagacttgaggtgtggagaggat | |
| | agagggcctgctaccttgagaacttcccactcagggcagcgacctctgtacacggtcatttcaagcacac | |
| | ctggtctcagtgttcattgtttatgttccccccgaccccaccctgggcacatacattttcctgctccatgagg | |
| | gatccttgtccctgccctccctgccctgtagagataggagttgctcttacttacttgatcaaatacacatcata | |
| | cttcccagcagagcggccagatttcctttgcttaagcttccgtgtccagccttcaggcagggtggggtcat | |
| | catacatgggtccccggtcacggatgatggagcgccgctgtttgggggaggcagaagcttccggcaca | |
| | gccggggggagcctgacccttctgatgtctctgctttgcctgcctctgcgggctcagcagagtggtggg | |
| | ctgatggctgcacgggctcatgcttgccctctttctcttctttcttatctttcttcacctttttaaacttgaggggtt | |
| | tgtccttgaggccctggaggtcctggtcttctgacttttcttccctgaagtgttaaacaagtatgtaagtatca | |
| | cagagaacatgccagtctgcagaacaagtgagcagaggcaggcttggacccccaccacatgcaaggc | |
| | cccaggccatcatacactcagatcttgatgtccttccccaggacaacagagactcgggccaagctggga | |
| | cccaaatatttctttaggaatttgggctgtgggtggtgctggccggtgtgcctgacctgcactgtggccacc | |
| | cacaagtctcctagcactgcaggagaatcagagccctttcctctgagcggaaacactgcggctcctgag | |
| | cataccaccccagcatgagtcgcacttcaagggactctgggctcgatggctgacaagaggccgtcgact | |
| | gctggaaagggagccaaatgggggaggggtctcctttgtgaaggccgggccttgggtgtgtttttggttg | |
| | cggccgcaaaaactagttattaatagtaatcaattacggggtcattagttcatagcccatatatggagttccg | |
| | cgttacataacttacggtaaatggcccgcctggctgaccgcccaacgacccccgcccattgacgtcaata | |
| | atgacgtatgttcccatagtaacgccaatagggactttccattgacgtcaatgggtggactatttacggtaa | |
| | actgcccacttggcagtacatcaagtgtatcatatgccaagtacgccccctattgacgtcaatgacggtaa | |
| | atggcccgcctggcattatgcccagtacatgaccttatgggactttcctacttggcagtacatctacgtatta | |
| | gtcatcgctattaccatgggtcgaggtgagccccacgttctgcttcactctccccatctcccccccctcccc | |
| | acccccaattttgtatttatttattttttaattattttgtgcagcgatgggggcggggggggggggggcgcgc | |
| | gccaggcggggcggggcggggcgaggggcggggggggcgaggcggagaggtgcggcggcag | |
| | ccaatcagagcggcgcgctccgaaagtttccttttatggcgaggcggcggcggcggcggccctataaa | |
| | aagcgaagcgcgcggggggggagtcgctgcgttgccttcgccccgtgccccgctccgcgccgcct | |
| | cgcgccgcccgccccggctctgactgaccgcgttactcccacaggtgagcggggggacggcccttct | |
| | cctccgggctgtaattagcgcttggtttaatgacggctcgtttcttttctgtggctgcgtgaaagccttaaag | |
| | ggctccgggagggccctttgtgcgggggggagcggctcggggggtgcgtgcgtgtgtgtgtgcgtgg | |
| | ggagcgccgcgtgcggcccgcgctgcccggcggctgtgagcgctgcgggcgcggcgcggggctttg | |
| | tgcgctccgcgtgtgcgcgaggggagcgcggccgggggcggtgccccgcggtgcgggggggctgc | |
| | gaggggaacaaaggctgcgtgcggggtgtgtgcgtgggggggtgagcagggggtgtgggcgcggc | |
| | ggtcgggctgtaacccccccctgcacccccctccccgagttgctgagcacggcccggcttcgggtgcg | |
| | gggctccgtgcggggcgtggcgcggggctcgccgtgccgggcggggggtggcggcaggtgggggt | |
| | gccgggcggggggggccgcctcgggccggggagggctcgggggaggggcgcggcggccccgg | |
| | agcgccggcggctgtcgaggcgcggcgagccgcagccattgccttttatggtaatcgtgcgagagggc | |
| | gcagggacttcctttgtcccaaatctggcggagccgaaatctgggaggcgccgccgcaccccctctagc | |
| | gggcgcgggcgaagcggtgcggcgccggcaggaaggaaatgggcggggagggccttcgtgcgtcg | |
| | ccgcgccgccgtccccttctccatctccagcctcggggctgccgcagggggacggctgccttcggggg | |
| | ggacggggcagggcggggttcggcttctggcgtgtgaccggcggctctagagcctctgctaaccatgtt | |
| | catgccttcttctttttcctacagctcctgggcaacgtgctggttgttgtgctgtctcatcattttggcaaagaat | |
| | tcgccaccatggtgagcaagggcgaggagctgttcaccggggggtgcccatcctggtcgagctggac | |
| | ggcgacgtaaacggccacaagttcagcgtgtccggcgagggcgagggcgatgccacctacggcaag | |
| | ctgaccctgaagttcatctgcaccaccggcaagctgcccgtgccctggcccaccctcgtgaccaccctg | |
| | acctacggcgtgcagtgcttcagccgctaccccgaccacatgaagcagcacgacttcttcaagtccgcc | |
| | atgcccgaaggctacgtccaggagcgcaccatcttcttcaaggacgacggcaactacaagacccgcgc | |
| | cgaggtgaagttcgagggcgacaccctggtgaaccgcatcgagctgaagggcatcgacttcaaggag | |
| | gacggcaacatcctggggcacaagctggagtacaactacaacagccacaacgtctatatcatggccga | |
| | caagcagaagaacggcatcaaggtgaacttcaagatccgccacaacatcgaggacggcagcgtgcag | |
| | ctcgccgaccactaccagcagaacacccccatcggcgacggccccgtgctgctgcccgacaaccacta | |
| | cctgagcacccagtccgccctgagcaaagaccccaacgagaagcgcgatcacatggtcctgctggagt | |
| | tcgtgaccgccgccgggatcactctcggcatggacgagctgtacaagtaagaattcactcctcaggtgca | |
| | ggctgcctatcagaaggtggtggctggtgtggccaatgccctggctcacaaataccactgagatctttttc | |
| | cctctgccaaaaattatggggacatcatgaagccccttgagcatctgacttctggctaataaaggaaattta | |
| | ttttcattgcaatagtgtgttggaattttttgtgtctctcactcggaaggacatatgggagggcaaatcatttaa | |
| | aacatcagaatgagtatttggtttagagtttggcaacatatgccatatgctggctgccatgaacaaaggtgg | |
| | ctataaagaggtcatcagtatatgaaacagccccctgctgtccattccttattccatagaaaagccttgactt | |
| | gaggttagattttttttatattttgttttgtgttatttttttctttaacatccctaaaattttccttacatgttttactagcc | |
| | agatttttcctcctctcctgactactcccagtcatagctgtccctcttctcttatgaagatccctcgacctgcag | |
| | atatccagcacagtggcggccgctcgagtctagagggcccgtttaaacccgctgatcagcctcgactgt | |
| | gccttctagttgccagccatctgttgtttgcccctcccccgtgccttccttgaccctggaaggtgccactccc | |
| | actgtcctttcctaataaaatgaggaaattgcatcgcattgtctgagtaggtgtcattctattctggggggtgg | |
| | ggtggggcaggacagcaagggggaggattgggaagacaatagcaggcatgctggggatgcggtgg | |
| | gctctatggcttctgaggcggaaagaaccagctggggctctagggggtatccccacgcgccctgtagcg | |
| | gcgcattaagcgcggcgggtgtggtggttacgcgcagcgtgaccgctacacttgccagcgccctagcg | |
| | cccgctcctttcgctttcttcccttcctttctcgccacgttcgccggctttccccgtcaagctctaaatcgggg | |
| | gctccctttagggttccgatttagtgctttacggcacctcgaccccaaaaaacttgattagggtgatggttca | |
| | cgtagtgggccatcgccctgatagacggtttttcgccctttgacgttggagtccacgttctttaatagtggac | |
| | tcttgttccaaactggaacaacactcaaccctatctcggtctattcttttgatttataagggattttgccgatttc | |
| | ggcctattggttaaaaaatgagctgatttaacaaaaatttaacgcgaattaattctgtggaatgtgtgtcagtt | |
| | agggtgtggaaagtccccaggctccccagcaggcagaagtatgcaaagcatgcatctcaattagtcagc | |
| | aaccaggtgtggaaagtccccaggctccccagcaggcagaagtatgcaaagcatgcatctcaattagtc | |
| | agcaaccatagtcccgcccctaactccgcccatcccgcccctaactccgcccagttccgcccattctccg | |
| | ccccatggctgactaattttttttatttatgcagaggccgaggccgcctctgcctctgagctattccagaagt | |
| | agtgaggaggcttttttggaggcctaggcttttgcaaaaagctcccgggagcttgtatatccattttcggatc | |
| | tgatcagcacgtgttgacaattaatcatcggcatagtatatcggcatagtataatacgacaaggtgaggaa | |
| | ctaaaccatggccaagttgaccagtgccgttccggtgctcaccgcgcgcgacgtcgccggagcggtcg | |
| | agttctggaccgaccggctcgggttctcccgggacttcgtggaggacgacttcgccggtgtggtccggg | |
| | acgacgtgaccctgttcatcagcgcggtccaggaccaggtggtgccggacaacaccctggcctgggtg | |
| | tgggtgcgcggcctggacgagctgtacgccgagtggtcggaggtcgtgtccacgaacttccgggacgc | |
| | ctccgggccggccatgaccgagatcggcgagcagccgtggggggggagttcgccctgcgcgaccc | |
| | ggccggcaactgcgtgcacttcgtggccgaggagcaggactgacacgtgctacgagatttcgattccac | |
| | cgccgccttctatgaaaggttgggcttcggaatcgttttccgggacgccggctggatgatcctccagcgc | |
| | ggggatctcatgctggagttcttcgcccaccccaacttgtttattgcagcttataatggttacaaataaagca | |
| | atagcatcacaaatttcacaaataaagcatttttttcactgcattctagttgtggtttgtccaaactcatcaatgt | |
| | atcttatcatgtctgtataccgtcgacctctagctagagcttggcgtaatcatggtcatagctgtttcctgtgtg | |
| | aaattgttatccgctcacaattccacacaacatacgagccggaagcataaagtgtaaagcctggggtgcc | |
| | taatgagtgagctaactcacattaattgcgttgcgctcactgcccgctttccagtcgggaaacctgtcgtgc | |
| | cagctgcattaatgaatcggccaacgcgcggggagaggcggtttgcgtattgggcgctcttccgcttcct | |
| | cgctcactgactcgctgcgctcggtcgttcggctgcggcgagcggtatcagctcactcaaaggcggtaat | |
| | acggttatccacagaatcaggggataacgcaggaaagaacatgtgagcaaaaggccagcaaaaggcc | |
| | aggaaccgtaaaaaggccgcgttgctggcgtttttccataggctccgcccccctgacgagcatcacaaa | |
| | aatcgacgctcaagtcagaggtggcgaaacccgacaggactataaagataccaggcgtttccccctgga | |
| | agctccctcgtgcgctctcctgttccgaccctgccgcttaccggatacctgtccgcctttctcccttcggga | |
| | agcgtggcgctttctcatagctcacgctgtaggtatctcagttcggtgtaggtcgttcgctccaagctgggc | |
| | tgtgtgcacgaaccccccgttcagcccgaccgctgcgccttatccggtaactatcgtcttgagtccaaccc | |
| | ggtaagacacgacttatcgccactggcagcagccactggtaacaggattagcagagcgaggtatgtag | |
| | gcggtgctacagagttcttgaagtggtggcctaactacggctacactagaagaacagtatttggtatctgc | |
| | gctctgctgaagccagttaccttcggaaaaagagttggtagctcttgatccggcaaacaaaccaccgctg | |
| | gtagcggtttttttgtttgcaagcagcagattacgcgcagaaaaaaaggatctcaagaagatcctttgatctt | |
| | ttctacggggtctgacgctcagtggaacgaaaactcacgttaagggattttggtcatgagattatcaaaaa | |
| | ggatcttcacctagatccttttaaattaaaaatgaagttttaaatcaatctaaagtatatatgagtaaacttggtc | |
| | tgacagttaccaatgcttaatcagtgaggcacctatctcagcgatctgtctatttcgttcatccatagttgcct | |
| | gactccccgtcgtgtagataactacgatacgggagggcttaccatctggccccagtgctgcaatgatacc | |
| | gcgagacccacgctcaccggctccagatttatcagcaataaaccagccagccggaagggccgagcgc | |
| | agaagtggtcctgcaactttatccgcctccatccagtctattaattgttgccgggaagctagagtaagtagtt | |
| | cgccagttaatagtttgcgcaacgttgttgccattgctacaggcatcgtggtgtcacgctcgtcgtttggtat | |
| | ggcttcattcagctccggttcccaacgatcaaggcgagttacatgatcccccatgttgtgcaaaaaagcgg | |
| | ttagctccttcggtcctccgatcgttgtcagaagtaagttggccgcagtgttatcactcatggttatggcagc | |
| | actgcataattctcttactgtcatgccatccgtaagatgcttttctgtgactggtgagtactcaaccaagtcatt | |
| | ctgagaatagtgtatgcggcgaccgagttgctcttgcccggcgtcaatacgggataataccgcgccacat | |
| | agcagaactttaaaagtgctcatcattggaaaacgttcttcggggcgaaaactctcaaggatcttaccgct | |
| | gttgagatccagttcgatgtaacccactcgtgcacccaactgatcttcagcatcttttactttcaccagcgttt | |
| | ctgggtgagcaaaaacaggaaggcaaaatgccgcaaaaaagggaataagggcgacacggaaatgttg | |
| | aatactcatactcttcctttttcaatattattgaagcatttatcagggttattgtctcatgagcggatacatatttg | |
| | aatgtatttagaaaaataaacaaataggggttccgcgcacatttccccgaaaagtgccacctgacgtc | |
| 5 | aaccaaaaacacacccaaggcccggccttcacaaaggagacccctcccccatttggctccctttccagc | Mecp2 |
| | agtcgacggcctcttgtcagccatcgagcccagagtcccttgaagtgcgactcatgctggggtggtatgc | |
| | tcaggagccgcagtgtttccgctcagaggaaagggctctgattctcctgcagtgctaggagacttgtggg | |
| | tggccacagtgcaggtcaggcacaccggccagcaccacccacagcccaaattcctaaagaaatatttgg | |
| | gtcccagcttggcccgagtctctgttgtcctggggaaggacatcaagatctgagtgtatgatggcctggg | |
| | gccttgcatgtggtgggggtccaagcctgcctctgctcacttgttctgcagactggcatgttctctgtgatac | |
| | ttacatacttgtttaacacttcagggaagaaaagtcagaagaccaggacctccagggcctcaaggacaaa | |
| | cccctcaagtttaaaaaggtgaagaaagataagaaagaagagaaagagggcaagcatgagcccgtgc | |
| | agccatcagcccaccactctgctgagcccgcagaggcaggcaaagcagagacatcagaagggtcag | |
| | gctccgccccggctgtgccggaagcttctgcctcccccaagcaaagaaggagtattatccgtgaccggg | |
| | gacccatgtatgatgaccccaccctgcctgaaggctggacacggaagcttaagcaaaggaaatctggcc | |
| | gctctgctgggaagtatgatgtgtatttgatcaagtaagtaagagcaactcctatctctacagggcaggga | |
| | gggcagggacaaggatccctcatggagcaggaaaatgtatgtgcccagggggggtcggggggaac | |
| | ataaacaatgaacactgagaccaggtgtgcttgaaatgaccgtgtacagaggtcgctgccctgagtggg | |
| | aagttctcaaggtagcaggccctctatcctctccacacctcaagtctttatctggggatggaatagctgcgg | |
| | aagcagaggaacttgcagagccaggggttcagaggggtgaagaagcatgtttcagttctgccttttaaat | |
| | gatcccaaaaaggttagcagttttcaaatgacatttgcagacagcctcatttaattccatgagaagggtgag | |
| | caaaggattatcttgttgaaactgattcctggagagactgagcaccgtacctgagttcaaacttgggaatgt | |
| | tctagatggtgactcaggcccaggcaccaaccagcagaatgggcctcagcctgacaacccttctgtacc | |
| | aggcctgactctttggttgctgaactttggagaggcctgggggggtcagcggcaggcagacgagtgagt | |
| | ggctttg | |

What is claimed is:

1. A magnetic-assisted nanoparticle delivery and gene editing system, comprising:
a magnetic core-shell nanoparticle (MCNP) having a magnetic core and a negatively-charged shell coated on the magnetic core;
an inner cationic polymer layer disposed on the surface of the negatively-charged shell;
a multi-plasmid layer disposed on the inner cationic polymer layer, the multi-plasmid layer comprising one or more polynucleotides, wherein the one or more polynucleotides are associated through electrostatic interactions with the inner cationic polymer layer; and
an outer cationic polymer layer disposed on the multi-plasmid layer, wherein the outer cationic polymer layer is configured to encapsulate and protect the multi-plasmid layer and improve intracellular delivery/releasing efficiency of the one or more polynucleotides in a cell.

2. The system of claim 1, wherein the magnetic core comprises ZnFe₂O₄.

3. The system of claim 1, wherein the negatively-charged shell is a silica shell.

4. The system of claim 1, wherein the inner cationic polymer layer or the outer cationic polymer layer comprises branched polyethyleneimine (PEI).

5. The system of claim 4, wherein: (i) the PEI is a tetramethylrhodamine (TRITC)-labeled PEI; and/or (ii) the PEI has a molecular weight of about 10 kDa.

6. The system of claim 1, wherein the inner cationic polymer layer and the outer cationic polymer layer comprise the same or different cationic polymers.

7. The system of claim 1, wherein the system has a polynucleotide:MCNP mass ratio of about 1:10 to about 1:20.

8. The system of claim 1, wherein the system has a polynucleotide:MCNP mass ratio of about 1:15.

9. The system of claim 1, wherein: (i) the magnetic core has a diameter of about 6 nm to about 10 nm; and/or (ii) the silica shell has a thickness of about 20 nm to about 30 nm.

10. The system of claim 1, wherein: (i) the MCNP has an average diameter of about 80 nm to about 110 nm; (ii) the MCNP has an average hydrodynamic size of about 95 nm to about 105 nm; and/or (iii) wherein the MCNP has a zeta potential of about 18 mV to about 28 mV.

11. The system of claim 1, wherein the one or more polynucleotides comprise a first polynucleotide and a second polynucleotide that is different from the first polynucleotide.

12. The system of claim 11, wherein the first polynucleotide or the second polynucleotide comprises a plasmid.

13. The system of claim 12, wherein the plasmid is linearized.

14. The system of claim 11, wherein: (i) the first polynucleotide comprises a CRISPR-associated (Cas) gene, wherein the Cas gene is selected from the group consisting of: Cas9 (Csn1), Cas12a (Cpf1), Cas13a (C2c2), and Cas13b (C2c6); (ii) the first polynucleotide further comprises a gRNA sequence comprising a crRNA and optionally a tracrRNA; and/or (iii) the one or more polynucleotides comprise a third polynucleotide comprising a gRNA sequence comprising a crRNA and optionally a tracrRNA.

15. The system of claim 11, wherein the second polynucleotide comprises a donor DNA.

16. The system of claim 15, wherein the donor DNA comprises a methyl CpG binding protein 2 (MECP2) gene.

17. The method of claim 1, wherein: (i) the cell comprises a plant, animal, or human cell; and/or (ii) the cell comprises a stem cell.

18. A composition comprising the system of claim 1.

19. A method of modifying a target sequence of interest in a cell, comprising delivering the system of claim 1 to a cell containing the target sequence.

20. A method of treating a disease of a subject caused by a genetic defect in a target sequence, comprising: administering the system of claim 1 to a cell containing the target sequence in a subject in need thereof.
